SEQUENCE LISTING (86 sequences)

SEQ ID NO: 1 - protein, 13 aa, Artificial Sequence
  MTD Amino Acid Sequence
KAMININKFLNQC

SEQ ID NO: 2 - DNA, 39 bp, Artificial Sequence
  MTD cDNA Sequence
aaggcgatgataaacataaacaagttcctgaaccagtgc

SEQ ID NO: 3 - protein, 448 aa, Artificial Sequence
  BoNT/A Light chain Amino Acid Sequence
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 4 - protein, 441 aa, Artificial Sequence
  BoNT/B Light chain Amino Acid Sequence
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVK

SEQ ID NO: 5 - protein, 449 aa, Artificial Sequence
  BoNT/C Light chain Amino Acid Sequence
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNK

SEQ ID NO: 6 - protein, 442 aa, Artificial Sequence
  BoNT/D Light chain Amino Acid Sequence
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTK

SEQ ID NO: 7 - protein, 422 aa, Artificial Sequence
  BoNT/E Light chain Amino Acid Sequence
MPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIR

SEQ ID NO: 8 - protein, 436 aa, Artificial Sequence
  BoNT/F Light chain Amino Acid Sequence
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRK

SEQ ID NO: 9 - protein, 441 aa, Artificial Sequence
  BoNT/G Light chain Amino Acid Sequence
MPVNIKFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYK

SEQ ID NO: 10 - DNA, 1347 bp, Artificial Sequence
  BoNT/A Light chain cDNA Sequence
atgccctttgtcaacaaacagttcaactacaaggacccagttaatggagtagacatcgcatatatcaagattcccaacgctggccagatgcaacccgttaaggcatttaaaatccataacaaaatctgggttatcccagagcgggataccttcaccaaccccgaggagggcgatctgaaccccccgccggaggcgaagcaggtcccagtgagctactacgatagcacctacctcagcaccgacaacgagaaggacaactacctcaaaggagtcacgaagttgttcgagagaatctactccacagacctcggccgcatgcttctaaccagcattgtgcgtggcattcccttttggggcggctctaccatcgacacagagctgaaggtgatagacaccaactgcatcaacgtaatccagcctgacggcagctaccgaagcgaggagcttaacctggtgatcatcggcccttccgccgatatcatccaattcgagtgcaagagcttcggccacgaggtcctgaacctcacccggaacggctatggaagcacccagtacataagattcagccctgacttcaccttcgggtttgaggagagcttggaggtcgacacaaaccccctgctgggagccgggaagttcgccactgacccagccgtgactctggcacacgagctgatccacgccggtcaccgcctgtacggcatagctataaacccaaacagggtgttcaaagtgaacaccaacgcttactatgaaatgagcggcctggaggtgagcttcgaggagctgagaacgttcgggggacatgatgctaaatttatcgacagcctgcaggagaacgagttcaggctgtactactacaataagttcaaggatatagcgagcactctgaacaaggccaagtccatcgtaggcactactgcatccctccagtatatgaagaatgtgttcaaagagaaatacctgctgagcgaggataccagcggtaagttcagcgtggataagcttaagttcgacaagctgtataagatgctcaccgaaatctacaccgaggataatttcgttaagttcttcaaggtcctgaaccggaagacctacctgaacttcgacaaggccgtgttcaagatcaacatcgtgcctaaagtgaactacaccatctacgacgggtttaacctgaggaacaccaacctggccgctaacttcaacgggcagaacacagagatcaacaacatgaatttcacgaagttgaagaacttcaccggactgtttgagttctacaaattgctgtgtgtgcgcgggatcatcactagcaagaccaagagccttgacaaaggctacaacaagtga

SEQ ID NO: 11 - DNA, 1323 bp, Artificial Sequence
  BoNT/B Light chain cDNA Sequence
atgccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaa

SEQ ID NO: 12 - DNA, 1347 bp, Artificial Sequence
  BoNT/C Light chain cDNA Sequence
atgccaataacaattaacaactttaattattcagatcctgttgataataaaaatattttatatttagatactcatttaaatacactagctaatgagcctgaaaaagcctttcgcattacaggaaatatatgggtaatacctgatagattttcaagaaattctaatccaaatttaaataaacctcctcgagttacaagccctaaaagtggttattatgatcctaattatttgagtactgattctgacaaagatacatttttaaaagaaattataaagttatttaaaagaattaattctagagaaataggagaagaattaatatatagactttcgacagatataccctttcctgggaataacaatactccaattaatacttttgattttgatgtagattttaacagtgttgatgttaaaactagacaaggtaacaactgggttaaaactggtagcataaatcctagtgttataataactggacctagagaaaacattatagatccagaaacttctacgtttaaattaactaacaatacttttgcggcacaagaaggatttggtgctttatcaataatttcaatatcacctagatttatgctaacatatagtaatgcaactaatgatgtaggagagggtagattttctaagtctgaattttgcatggatccaatactaattttaatgcatgaacttaatcatgcaatgcataatttatatggaatagctataccaaatgatcaaacaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatctatagctaaaagacttaatagtataactactgcaaatccttcaagctttaataaatatataggggaatataaacagaaacttattagaaagtatagattcgtagtagaatcttcaggtgaagttacagtaaatcgtaataagtttgttgagttatataatgaacttacacaaatatttacagaatttaactacgctaaaatatataatgtacaaaataggaaaatatatctttcaaatgtatatactccggttacggcgaatatattagacgataatgtttatgatatacaaaatggatttaatatacctaaaagtaatttaaatgtactatttatgggtcaaaatttatctcgaaatccagcattaagaaaagtcaatcctgaaaatatgctttatttatttacaaaattttgtcataaagcaatagatggtagatcattatataataaa

SEQ ID NO: 13 - DNA, 1326 bp, Artificial Sequence
  BoNT/D Light chain cDNA Sequence
atgacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcccagacctacttcaaagtatcaaagttattatgatcctagttatttatctactgatgaacaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattatatggaataaatataccatctgataaaaggattcgtccacaagttagcgagggatttttctctcaagatggacccaacgtacaatttgaggaattatatacatttggaggattagatgttgaaataatacctcaaattgaaagatcacaattaagagaaaaagcattaggtcactataaagatatagcgaaaagacttaataatattaataaaactattccttctagttggattagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaggcattatctacctgtatttgcaaatatattagatgataatatttatactataagagatggttttaatttaacaaataaaggttttaatatagaaaattcgggtcagaatatagaaaggaatcctgcactacaaaagcttagttcagaaagtgtagtagatttatttacaaaagtatgtttaagattaacaaaa

SEQ ID NO: 14 - DNA, 1266 bp, Artificial Sequence
  BoNT/E Light chain cDNA Sequence
atgccaacaattaatagttttaattataatgatcctgttaataatagaacaattttatatattaaaccaggcggttgtcaacaattttataaatcatttaatattatgaaaaatatttggataattccagagagaaatgtaattggtacaattccccaagattttcttccgcctacttcattgaaaaatggagatagtagttattatgaccctaattatttacaaagtgatcaagaaaaggataaatttttaaaaatagtcacaaaaatatttaatagaataaatgataatctttcaggaaggattttattagaagaactgtcaaaagctaatccatatttaggaaatgataatactccagatggtgacttcattattaatgatgcatcagcagttccaattcaattctcaaatggtagccaaagcatactattacctaatgttattataatgggagcagagcctgatttatttgaaactaacagttccaatatttctctaagaaataattatatgccaagcaatcacggttttggatcaatagctatagtaacattctcacctgaatattcttttagatttaaagataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataagg

SEQ ID NO: 15 - DNA, 1308 bp, Artificial Sequence
  BoNT/F Light chain cDNA Sequence
atgccagttgcaataaatagttttaattataatgaccctgttaatgatgatacaattttatacatgcagataccatatgaagaaaaaagtaaaaaatattataaagcttttgagattatgcgtaatgtttggataattcctgagagaaatacaataggaacgaatcctagtgattttgatccaccggcttcattaaagaacggaagcagtgcttattatgatcctaattatttaaccactgatgctgaaaaagatagatatttaaaaacaacgataaaattatttaagagaattaatagtaatcctgcagggaaagttttgttacaagaaatatcatatgctaaaccatatttaggaaatgaccacacgccaattgatgaattctctccagttactagaactacaagtgttaatataaaattatcaactaatgttgaaagttcaatgttattgaatcttcttgtattgggagcaggacctgatatatttgaaagttgttgttaccccgttagaaaactaatagatccagatgtagtttatgatccaagtaattatggttttggatcaattaatatcgtgacattttcacctgagtatgaatatacttttaatgatattagtggagggcataatagtagtacagaatcatttattgcagatcctgcaatttcactagctcatgaattgatacatgcactgcatggattatacggggctaggggagttacttatgaagagactatagaagtaaagcaagcacctcttatgatagccgaaaaacccataaggctagaagaatttttaacctttggaggtcaggatttaaatattattactagtgctatgaaggaaaaaatatataacaatcttttagctaactatgaaaaaatagctactagacttagtgaagttaatagtgctcctcctgaatatgatattaatgaatataaagattattttcaatggaagtatgggctagataaaaatgctgatggaagttatactgtaaatgaaaataaatttaatgaaatttataaaaaattatatagttttacagagagtgacttagcaaataaatttaaagtaaaatgtagaaatacttattttattaaatatgaatttttaaaagttccaaatttgttagatgatgatatttatactgtatcagaggggtttaatataggtaatttagcagtaaacaatcgcggacaaagtataaagttaaatcctaaaattattgattccattccagataaaggtctagtagaaaagatcgttaaattttgtaagagcgttattcctagaaaa

SEQ ID NO: 16 - DNA, 1326 bp, Artificial Sequence
  BoNT/G Light chain cDNA Sequence
  MISC_FEATURE (20)..(20): n = a, t, c, or g
atgccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaa

SEQ ID NO: 17 - protein, 468 aa, Artificial Sequence
  BoNT/A Light chain Amino Acid Sequence with hexahistidine
MGSSHHHHHHSSGLVPRGSHMPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 18 - protein, 461 aa, Artificial Sequence
  BoNT/B Light chain Amino Acid Sequence with hexahistidine
MGSSHHHHHHSSGLVPRGSHMPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVK

SEQ ID NO: 19 - protein, 469 aa, Artificial Sequence
  BoNT/C Light chain Amino Acid Sequence with hexahistidine
MGSSHHHHHHSSGLVPRGSHMPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNK

SEQ ID NO: 20 - protein, 462 aa, Artificial Sequence
  BoNT/D Light chain Amino Acid Sequence with hexahistidine
MGSSHHHHHHSSGLVPRGSHMTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTK

SEQ ID NO: 21 - protein, 442 aa, Artificial Sequence
  BoNT/E Light chain Amino Acid Sequence with hexahistidine
MGSSHHHHHHSSGLVPRGSHMPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIR

SEQ ID NO: 22 - protein, 456 aa, Artificial Sequence
  BoNT/F Light chain Amino Acid Sequence with hexahistidine
MGSSHHHHHHSSGLVPRGSHMPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRK

SEQ ID NO: 23 - protein, 461 aa, Artificial Sequence
  BoNT/G Light chain Amino Acid Sequence with hexahistidine
MGSSHHHHHHSSGLVPRGSHMPVNIKFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYK

SEQ ID NO: 24 - DNA, 1437 bp, Artificial Sequence
  BoNT/A Light chain cDNA Sequence with hexahistidine
atgggcagcagccatcatcatcatcatcacagcagcggcctggtgccgcgcggcagccatatgatgccctttgtcaacaaacagttcaactacaaggacccagttaatggagtagacatcgcatatatcaagattcccaacgctggccagatgcaacccgttaaggcatttaaaatccataacaaaatctgggttatcccagagcgggataccttcaccaaccccgaggagggcgatctgaaccccccgccggaggcgaagcaggtcccagtgagctactacgatagcacctacctcagcaccgacaacgagaaggacaactacctcaaaggagtcacgaagttgttcgagagaatctactccacagacctcggccgcatgcttctaaccagcattgtgcgtggcattcccttttggggcggctctaccatcgacacagagctgaaggtgatagacaccaactgcatcaacgtaatccagcctgacggcagctaccgaagcgaggagcttaacctggtgatcatcggcccttccgccgatatcatccaattcgagtgcaagagcttcggccacgaggtcctgaacctcacccggaacggctatggaagcacccagtacataagattcagccctgacttcaccttcgggtttgaggagagcttggaggtcgacacaaaccccctgctgggagccgggaagttcgccactgacccagccgtgactctggcacacgagctgatccacgccggtcaccgcctgtacggcatagctataaacccaaacagggtgttcaaagtgaacaccaacgcttactatgaaatgagcggcctggaggtgagcttcgaggagctgagaacgttcgggggacatgatgctaaatttatcgacagcctgcaggagaacgagttcaggctgtactactacaataagttcaaggatatagcgagcactctgaacaaggccaagtccatcgtaggcactactgcatccctccagtatatgaagaatgtgttcaaagagaaatacctgctgagcgaggataccagcggtaagttcagcgtggataagcttaagttcgacaagctgtataagatgctcaccgaaatctacaccgaggataatttcgttaagttcttcaaggtcctgaaccggaagacctacctgaacttcgacaaggccgtgttcaagatcaacatcgtgcctaaagtgaactacaccatctacgacgggtttaacctgaggaacaccaacctggccgctaacttcaacgggcagaacacagagatcaacaacatgaatttcacgaagttgaagaacttcaccggactgtttgagttctacaaattgctgtgtgtgcgcgggatcatcactagcaagaccaagagccttgacaaaggctacaacaagtgactcgagcaccaccaccaccaccactga

SEQ ID NO: 25 - DNA, 1413 bp, Artificial Sequence
  BoNT/B Light chain cDNA Sequence with hexahistidine
atgggcagcagccatcatcatcatcatcacagcagcggcctggtgccgcgcggcagccatatgatgccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaactcgagcaccaccaccaccaccactga

SEQ ID NO: 26 - DNA, 1437 bp, Artificial Sequence
  BoNT/C Light chain cDNA Sequence with hexahistidine
atgggcagcagccatcatcatcatcatcacagcagcggcctggtgccgcgcggcagccatatgatgccaataacaattaacaactttaattattcagatcctgttgataataaaaatattttatatttagatactcatttaaatacactagctaatgagcctgaaaaagcctttcgcattacaggaaatatatgggtaatacctgatagattttcaagaaattctaatccaaatttaaataaacctcctcgagttacaagccctaaaagtggttattatgatcctaattatttgagtactgattctgacaaagatacatttttaaaagaaattataaagttatttaaaagaattaattctagagaaataggagaagaattaatatatagactttcgacagatataccctttcctgggaataacaatactccaattaatacttttgattttgatgtagattttaacagtgttgatgttaaaactagacaaggtaacaactgggttaaaactggtagcataaatcctagtgttataataactggacctagagaaaacattatagatccagaaacttctacgtttaaattaactaacaatacttttgcggcacaagaaggatttggtgctttatcaataatttcaatatcacctagatttatgctaacatatagtaatgcaactaatgatgtaggagagggtagattttctaagtctgaattttgcatggatccaatactaattttaatgcatgaacttaatcatgcaatgcataatttatatggaatagctataccaaatgatcaaacaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatctatagctaaaagacttaatagtataactactgcaaatccttcaagctttaataaatatataggggaatataaacagaaacttattagaaagtatagattcgtagtagaatcttcaggtgaagttacagtaaatcgtaataagtttgttgagttatataatgaacttacacaaatatttacagaatttaactacgctaaaatatataatgtacaaaataggaaaatatatctttcaaatgtatatactccggttacggcgaatatattagacgataatgtttatgatatacaaaatggatttaatatacctaaaagtaatttaaatgtactatttatgggtcaaaatttatctcgaaatccagcattaagaaaagtcaatcctgaaaatatgctttatttatttacaaaattttgtcataaagcaatagatggtagatcattatataataaactcgagcaccaccaccaccaccactga

SEQ ID NO: 27 - DNA, 1416 bp, Artificial Sequence
  BoNT/D Light chain cDNA Sequence with hexahistidine
atgggcagcagccatcatcatcatcatcacagcagcggcctggtgccgcgcggcagccatatgatgacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcccagacctacttcaaagtatcaaagttattatgatcctagttatttatctactgatgaacaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattatatggaataaatataccatctgataaaaggattcgtccacaagttagcgagggatttttctctcaagatggacccaacgtacaatttgaggaattatatacatttggaggattagatgttgaaataatacctcaaattgaaagatcacaattaagagaaaaagcattaggtcactataaagatatagcgaaaagacttaataatattaataaaactattccttctagttggattagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaggcattatctacctgtatttgcaaatatattagatgataatatttatactataagagatggttttaatttaacaaataaaggttttaatatagaaaattcgggtcagaatatagaaaggaatcctgcactacaaaagcttagttcagaaagtgtagtagatttatttacaaaagtatgtttaagattaacaaaactcgagcaccaccaccaccaccactga

SEQ ID NO: 28 - DNA, 1356 bp, Artificial Sequence
  BoNT/E Light chain cDNA Sequence with hexahistidine
atgggcagcagccatcatcatcatcatcacagcagcggcctggtgccgcgcggcagccatatgatgccaacaattaatagttttaattataatgatcctgttaataatagaacaattttatatattaaaccaggcggttgtcaacaattttataaatcatttaatattatgaaaaatatttggataattccagagagaaatgtaattggtacaattccccaagattttcttccgcctacttcattgaaaaatggagatagtagttattatgaccctaattatttacaaagtgatcaagaaaaggataaatttttaaaaatagtcacaaaaatatttaatagaataaatgataatctttcaggaaggattttattagaagaactgtcaaaagctaatccatatttaggaaatgataatactccagatggtgacttcattattaatgatgcatcagcagttccaattcaattctcaaatggtagccaaagcatactattacctaatgttattataatgggagcagagcctgatttatttgaaactaacagttccaatatttctctaagaaataattatatgccaagcaatcacggttttggatcaatagctatagtaacattctcacctgaatattcttttagatttaaagataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggctcgagcaccaccaccaccaccactga

SEQ ID NO: 29 - DNA, 1398 bp, Artificial Sequence
  BoNT/F Light chain cDNA Sequence with hexahistidine
atgggcagcagccatcatcatcatcatcacagcagcggcctggtgccgcgcggcagccatatgatgccagttgcaataaatagttttaattataatgaccctgttaatgatgatacaattttatacatgcagataccatatgaagaaaaaagtaaaaaatattataaagcttttgagattatgcgtaatgtttggataattcctgagagaaatacaataggaacgaatcctagtgattttgatccaccggcttcattaaagaacggaagcagtgcttattatgatcctaattatttaaccactgatgctgaaaaagatagatatttaaaaacaacgataaaattatttaagagaattaatagtaatcctgcagggaaagttttgttacaagaaatatcatatgctaaaccatatttaggaaatgaccacacgccaattgatgaattctctccagttactagaactacaagtgttaatataaaattatcaactaatgttgaaagttcaatgttattgaatcttcttgtattgggagcaggacctgatatatttgaaagttgttgttaccccgttagaaaactaatagatccagatgtagtttatgatccaagtaattatggttttggatcaattaatatcgtgacattttcacctgagtatgaatatacttttaatgatattagtggagggcataatagtagtacagaatcatttattgcagatcctgcaatttcactagctcatgaattgatacatgcactgcatggattatacggggctaggggagttacttatgaagagactatagaagtaaagcaagcacctcttatgatagccgaaaaacccataaggctagaagaatttttaacctttggaggtcaggatttaaatattattactagtgctatgaaggaaaaaatatataacaatcttttagctaactatgaaaaaatagctactagacttagtgaagttaatagtgctcctcctgaatatgatattaatgaatataaagattattttcaatggaagtatgggctagataaaaatgctgatggaagttatactgtaaatgaaaataaatttaatgaaatttataaaaaattatatagttttacagagagtgacttagcaaataaatttaaagtaaaatgtagaaatacttattttattaaatatgaatttttaaaagttccaaatttgttagatgatgatatttatactgtatcagaggggtttaatataggtaatttagcagtaaacaatcgcggacaaagtataaagttaaatcctaaaattattgattccattccagataaaggtctagtagaaaagatcgttaaattttgtaagagcgttattcctagaaaactcgagcaccaccaccaccaccactga

SEQ ID NO: 30 - DNA, 1416 bp, Artificial Sequence
  BoNT/G Light chain cDNA Sequence with hexahistidine
  MISC_FEATURE (83)..(83): n = a, t, c, or g
atgggcagcagccatcatcatcatcatcacagcagcggcctggtgccgcgcggcagccatatgatgccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaactcgagcaccaccaccaccaccactga

SEQ ID NO: 31 - protein, 461 aa, Artificial Sequence
  MTD-BoNT/A Light chain Amino Acid Sequence
MKAMININKFLNQCPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 32 - protein, 454 aa, Artificial Sequence
  MTD-BoNT/B Light chain Amino Acid Sequence
MKAMININKFLNQCPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVK

SEQ ID NO: 33 - protein, 462 aa, Artificial Sequence
  MTD-BoNT/C Light chain Amino Acid Sequence
MKAMININKFLNQCPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNK

SEQ ID NO: 34 - protein, 455 aa, Artificial Sequence
  MTD-BoNT/D Light chain Amino Acid Sequence
MKAMININKFLNQCTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTK

SEQ ID NO: 35 - protein, 435 aa, Artificial Sequence
  MTD-BoNT/E Light chain Amino Acid Sequence
MKAMININKFLNQCPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIR

SEQ ID NO: 36 - protein, 449 aa, Artificial Sequence
  MTD-BoNT/F Light chain Amino Acid Sequence
MKAMININKFLNQCPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRK

SEQ ID NO: 37 - protein, 454 aa, Artificial Sequence
  MTD-BoNT/G Light chain Amino Acid Sequence
MKAMININKFLNQCPVNIKFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYK

SEQ ID NO: 38 - protein, 461 aa, Artificial Sequence
  BoNT/A Light chain-MTDr Amino Acid Sequence
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKCQNLFKNINIMAK

SEQ ID NO: 39 - protein, 454 aa, Artificial Sequence
  BoNT/B Light chain-MTDr Amino Acid Sequence
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKCQNLFKNINIMAK

SEQ ID NO: 40 - protein, 462 aa, Artificial Sequence
  BoNT/C Light chain-MTDr Amino Acid Sequence
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKCQNLFKNINIMAK

SEQ ID NO: 41 - protein, 455 aa, Artificial Sequence
  BoNT/D Light chain-MTDr Amino Acid Sequence
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKCQNLFKNINIMAK

SEQ ID NO: 42 - protein, 435 aa, Artificial Sequence
  BoNT/E Light chain-MTDr Amino Acid Sequence
MPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRCQNLFKNINIMAK

SEQ ID NO: 43 - protein, 449 aa, Artificial Sequence
  BoNT/F Light chain-MTDr Amino Acid Sequence
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKCQNLFKNINIMAK

SEQ ID NO: 44 - protein, 454 aa, Artificial Sequence
  BoNT/G Light chain-MTDr Amino Acid Sequence
MPVNIKFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKCQNLFKNINIMAK

SEQ ID NO: 45 - protein, 469 aa, Artificial Sequence
  MTD-BoNT/A Light chain Amino Acid Sequence with hexahistidine
MKAMININKFLNQCPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKLEHHHHHH

SEQ ID NO: 46 - protein, 462 aa, Artificial Sequence
  MTD-BoNT/B Light chain Amino Acid Sequence with hexahistidine
MKAMININKFLNQCPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKLEHHHHHH

SEQ ID NO: 47 - protein, 470 aa, Artificial Sequence
  MTD-BoNT/C Light chain Amino Acid Sequence with hexahistidine
MKAMININKFLNQCPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKLEHHHHHH

SEQ ID NO: 48 - protein, 463 aa, Artificial Sequence
  MTD-BoNT/D Light chain Amino Acid Sequence with hexahistidine
MKAMININKFLNQCTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKLEHHHHHH

SEQ ID NO: 49 - protein, 443 aa, Artificial Sequence
  MTD-BoNT/E Light chain Amino Acid Sequence with hexahistidine
MKAMININKFLNQCPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRLEHHHHHH

SEQ ID NO: 50 - protein, 457 aa, Artificial Sequence
  MTD-BoNT/F Light chain Amino Acid Sequence with hexahistidine
MKAMININKFLNQCPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKLEHHHHHH

SEQ ID NO: 51 - protein, 462 aa, Artificial Sequence
  MTD-BoNT/G Light chain Amino Acid Sequence with hexahistidine
MKAMININKFLNQCPVNIKFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKLEHHHHHH

SEQ ID NO: 52 - protein, 481 aa, Artificial Sequence
  BoNT/A Light chain-MTDr Amino Acid Sequence with hexahistidine
MGSSHHHHHHSSGLVPRGSHMPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKCQNLFKNINIMAK

SEQ ID NO: 53 - protein, 474 aa, Artificial Sequence
  BoNT/B Light chain-MTDr Amino Acid Sequence with hexahistidine
MGSSHHHHHHSSGLVPRGSHMPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKCQNLFKNINIMAK

SEQ ID NO: 54 - protein, 482 aa, Artificial Sequence
  BoNT/C Light chain-MTDr Amino Acid Sequence with hexahistidine
MGSSHHHHHHSSGLVPRGSHMPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKCQNLFKNINIMAK

SEQ ID NO: 55 - protein, 475 aa, Artificial Sequence
  BoNT/D Light chain-MTDr Amino Acid Sequence with hexahistidine
MGSSHHHHHHSSGLVPRGSHMTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKCQNLFKNINIMAK

SEQ ID NO: 56 - protein, 455 aa, Artificial Sequence
  BoNT/E Light chain-MTDr Amino Acid Sequence with hexahistidine
MGSSHHHHHHSSGLVPRGSHMPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRCQNLFKNINIMAK

SEQ ID NO: 57 - protein, 469 aa, Artificial Sequence
  BoNT/F Light chain-MTDr Amino Acid Sequence with hexahistidine
MGSSHHHHHHSSGLVPRGSHMPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKCQNLFKNINIMAK

SEQ ID NO: 58 - protein, 474 aa, Artificial Sequence
  BoNT/G Light chain-MTDr Amino Acid Sequence with hexahistidine
MGSSHHHHHHSSGLVPRGSHMPVNIKFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKCQNLFKNINIMAK

SEQ ID NO: 59 - DNA, 1386 bp, Artificial Sequence
  MTD-BoNT/A Light chain cDNA Sequence
atgaaggccatgatcaatattaacaagttcttaaatcaatgtccctttgtcaacaaacagttcaactacaaggacccagttaatggagtagacatcgcatatatcaagattcccaacgctggccagatgcaacccgttaaggcatttaaaatccataacaaaatctgggttatcccagagcgggataccttcaccaaccccgaggagggcgatctgaaccccccgccggaggcgaagcaggtcccagtgagctactacgatagcacctacctcagcaccgacaacgagaaggacaactacctcaaaggagtcacgaagttgttcgagagaatctactccacagacctcggccgcatgcttctaaccagcattgtgcgtggcattcccttttggggcggctctaccatcgacacagagctgaaggtgatagacaccaactgcatcaacgtaatccagcctgacggcagctaccgaagcgaggagcttaacctggtgatcatcggcccttccgccgatatcatccaattcgagtgcaagagcttcggccacgaggtcctgaacctcacccggaacggctatggaagcacccagtacataagattcagccctgacttcaccttcgggtttgaggagagcttggaggtcgacacaaaccccctgctgggagccgggaagttcgccactgacccagccgtgactctggcacacgagctgatccacgccggtcaccgcctgtacggcatagctataaacccaaacagggtgttcaaagtgaacaccaacgcttactatgaaatgagcggcctggaggtgagcttcgaggagctgagaacgttcgggggacatgatgctaaatttatcgacagcctgcaggagaacgagttcaggctgtactactacaataagttcaaggatatagcgagcactctgaacaaggccaagtccatcgtaggcactactgcatccctccagtatatgaagaatgtgttcaaagagaaatacctgctgagcgaggataccagcggtaagttcagcgtggataagcttaagttcgacaagctgtataagatgctcaccgaaatctacaccgaggataatttcgttaagttcttcaaggtcctgaaccggaagacctacctgaacttcgacaaggccgtgttcaagatcaacatcgtgcctaaagtgaactacaccatctacgacgggtttaacctgaggaacaccaacctggccgctaacttcaacgggcagaacacagagatcaacaacatgaatttcacgaagttgaagaacttcaccggactgtttgagttctacaaattgctgtgtgtgcgcgggatcatcactagcaagaccaagagccttgacaaaggctacaacaagtga

SEQ ID NO: 60 - DNA, 1362 bp, Artificial Sequence
  MTD-BoNT/B Light chain cDNA Sequence
atgaaggccatgatcaatattaacaagttcttaaatcaatgtccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaa

SEQ ID NO: 61 - DNA, 1386 bp, Artificial Sequence
  MTD-BoNT/C Light chain cDNA Sequence
atgaaggccatgatcaatattaacaagttcttaaatcaatgtccaataacaattaacaactttaattattcagatcctgttgataataaaaatattttatatttagatactcatttaaatacactagctaatgagcctgaaaaagcctttcgcattacaggaaatatatgggtaatacctgatagattttcaagaaattctaatccaaatttaaataaacctcctcgagttacaagccctaaaagtggttattatgatcctaattatttgagtactgattctgacaaagatacatttttaaaagaaattataaagttatttaaaagaattaattctagagaaataggagaagaattaatatatagactttcgacagatataccctttcctgggaataacaatactccaattaatacttttgattttgatgtagattttaacagtgttgatgttaaaactagacaaggtaacaactgggttaaaactggtagcataaatcctagtgttataataactggacctagagaaaacattatagatccagaaacttctacgtttaaattaactaacaatacttttgcggcacaagaaggatttggtgctttatcaataatttcaatatcacctagatttatgctaacatatagtaatgcaactaatgatgtaggagagggtagattttctaagtctgaattttgcatggatccaatactaattttaatgcatgaacttaatcatgcaatgcataatttatatggaatagctataccaaatgatcaaacaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatctatagctaaaagacttaatagtataactactgcaaatccttcaagctttaataaatatataggggaatataaacagaaacttattagaaagtatagattcgtagtagaatcttcaggtgaagttacagtaaatcgtaataagtttgttgagttatataatgaacttacacaaatatttacagaatttaactacgctaaaatatataatgtacaaaataggaaaatatatctttcaaatgtatatactccggttacggcgaatatattagacgataatgtttatgatatacaaaatggatttaatatacctaaaagtaatttaaatgtactatttatgggtcaaaatttatctcgaaatccagcattaagaaaagtcaatcctgaaaatatgctttatttatttacaaaattttgtcataaagcaatagatggtagatcattatataataaa

SEQ ID NO: 62 - DNA, 1365 bp, Artificial Sequence
  MTD-BoNT/D Light chain cDNA Sequence
atgaaggccatgatcaatattaacaagttcttaaatcaatgtacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcccagacctacttcaaagtatcaaagttattatgatcctagttatttatctactgatgaacaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattatatggaataaatataccatctgataaaaggattcgtccacaagttagcgagggatttttctctcaagatggacccaacgtacaatttgaggaattatatacatttggaggattagatgttgaaataatacctcaaattgaaagatcacaattaagagaaaaagcattaggtcactataaagatatagcgaaaagacttaataatattaataaaactattccttctagttggattagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaggcattatctacctgtatttgcaaatatattagatgataatatttatactataagagatggttttaatttaacaaataaaggttttaatatagaaaattcgggtcagaatatagaaaggaatcctgcactacaaaagcttagttcagaaagtgtagtagatttatttacaaaagtatgtttaagattaacaaaa

SEQ ID NO: 63 - DNA, 1305 bp, Artificial Sequence
  MTD-BoNT/E Light chain cDNA Sequence
atgaaggccatgatcaatattaacaagttcttaaatcaatgtccaacaattaatagttttaattataatgatcctgttaataatagaacaattttatatattaaaccaggcggttgtcaacaattttataaatcatttaatattatgaaaaatatttggataattccagagagaaatgtaattggtacaattccccaagattttcttccgcctacttcattgaaaaatggagatagtagttattatgaccctaattatttacaaagtgatcaagaaaaggataaatttttaaaaatagtcacaaaaatatttaatagaataaatgataatctttcaggaaggattttattagaagaactgtcaaaagctaatccatatttaggaaatgataatactccagatggtgacttcattattaatgatgcatcagcagttccaattcaattctcaaatggtagccaaagcatactattacctaatgttattataatgggagcagagcctgatttatttgaaactaacagttccaatatttctctaagaaataattatatgccaagcaatcacggttttggatcaatagctatagtaacattctcacctgaatattcttttagatttaaagataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataagg

SEQ ID NO: 64 - DNA, 1347 bp, Artificial Sequence
  MTD-BoNT/F Light chain cDNA Sequence
atgaaggccatgatcaatattaacaagttcttaaatcaatgtccagttgcaataaatagttttaattataatgaccctgttaatgatgatacaattttatacatgcagataccatatgaagaaaaaagtaaaaaatattataaagcttttgagattatgcgtaatgtttggataattcctgagagaaatacaataggaacgaatcctagtgattttgatccaccggcttcattaaagaacggaagcagtgcttattatgatcctaattatttaaccactgatgctgaaaaagatagatatttaaaaacaacgataaaattatttaagagaattaatagtaatcctgcagggaaagttttgttacaagaaatatcatatgctaaaccatatttaggaaatgaccacacgccaattgatgaattctctccagttactagaactacaagtgttaatataaaattatcaactaatgttgaaagttcaatgttattgaatcttcttgtattgggagcaggacctgatatatttgaaagttgttgttaccccgttagaaaactaatagatccagatgtagtttatgatccaagtaattatggttttggatcaattaatatcgtgacattttcacctgagtatgaatatacttttaatgatattagtggagggcataatagtagtacagaatcatttattgcagatcctgcaatttcactagctcatgaattgatacatgcactgcatggattatacggggctaggggagttacttatgaagagactatagaagtaaagcaagcacctcttatgatagccgaaaaacccataaggctagaagaatttttaacctttggaggtcaggatttaaatattattactagtgctatgaaggaaaaaatatataacaatcttttagctaactatgaaaaaatagctactagacttagtgaagttaatagtgctcctcctgaatatgatattaatgaatataaagattattttcaatggaagtatgggctagataaaaatgctgatggaagttatactgtaaatgaaaataaatttaatgaaatttataaaaaattatatagttttacagagagtgacttagcaaataaatttaaagtaaaatgtagaaatacttattttattaaatatgaatttttaaaagttccaaatttgttagatgatgatatttatactgtatcagaggggtttaatataggtaatttagcagtaaacaatcgcggacaaagtataaagttaaatcctaaaattattgattccattccagataaaggtctagtagaaaagatcgttaaattttgtaagagcgttattcctagaaaa

SEQ ID NO: 65 - DNA, 1365 bp, Artificial Sequence
  MTD-BoNT/G Light chain cDNA Sequence
  MISC_FEATURE (59)..(59): n = a, t, c, or g
atgaaggccatgatcaatattaacaagttcttaaatcaatgtccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaa

SEQ ID NO: 66 - DNA, 1386 bp, Artificial Sequence
  BoNT/A Light chain-MTDr cDNA Sequence
atgccctttgtcaacaaacagttcaactacaaggacccagttaatggagtagacatcgcatatatcaagattcccaacgctggccagatgcaacccgttaaggcatttaaaatccataacaaaatctgggttatcccagagcgggataccttcaccaaccccgaggagggcgatctgaaccccccgccggaggcgaagcaggtcccagtgagctactacgatagcacctacctcagcaccgacaacgagaaggacaactacctcaaaggagtcacgaagttgttcgagagaatctactccacagacctcggccgcatgcttctaaccagcattgtgcgtggcattcccttttggggcggctctaccatcgacacagagctgaaggtgatagacaccaactgcatcaacgtaatccagcctgacggcagctaccgaagcgaggagcttaacctggtgatcatcggcccttccgccgatatcatccaattcgagtgcaagagcttcggccacgaggtcctgaacctcacccggaacggctatggaagcacccagtacataagattcagccctgacttcaccttcgggtttgaggagagcttggaggtcgacacaaaccccctgctgggagccgggaagttcgccactgacccagccgtgactctggcacacgagctgatccacgccggtcaccgcctgtacggcatagctataaacccaaacagggtgttcaaagtgaacaccaacgcttactatgaaatgagcggcctggaggtgagcttcgaggagctgagaacgttcgggggacatgatgctaaatttatcgacagcctgcaggagaacgagttcaggctgtactactacaataagttcaaggatatagcgagcactctgaacaaggccaagtccatcgtaggcactactgcatccctccagtatatgaagaatgtgttcaaagagaaatacctgctgagcgaggataccagcggtaagttcagcgtggataagcttaagttcgacaagctgtataagatgctcaccgaaatctacaccgaggataatttcgttaagttcttcaaggtcctgaaccggaagacctacctgaacttcgacaaggccgtgttcaagatcaacatcgtgcctaaagtgaactacaccatctacgacgggtttaacctgaggaacaccaacctggccgctaacttcaacgggcagaacacagagatcaacaacatgaatttcacgaagttgaagaacttcaccggactgtttgagttctacaaattgctgtgtgtgcgcgggatcatcactagcaagaccaagagccttgacaaaggctacaacaagtgatgtcaaaatttattcaagaacattaatatcatggccaag

SEQ ID NO: 67 - DNA, 1362 bp, Artificial Sequence
  BoNT/B Light chain-MTDr cDNA Sequence
atgccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaatgtcaaaatttattcaagaacattaatatcatggccaag

SEQ ID NO: 68 - DNA, 1386 bp, Artificial Sequence
  BoNT/C Light chain-MTDr cDNA Sequence
atgccaataacaattaacaactttaattattcagatcctgttgataataaaaatattttatatttagatactcatttaaatacactagctaatgagcctgaaaaagcctttcgcattacaggaaatatatgggtaatacctgatagattttcaagaaattctaatccaaatttaaataaacctcctcgagttacaagccctaaaagtggttattatgatcctaattatttgagtactgattctgacaaagatacatttttaaaagaaattataaagttatttaaaagaattaattctagagaaataggagaagaattaatatatagactttcgacagatataccctttcctgggaataacaatactccaattaatacttttgattttgatgtagattttaacagtgttgatgttaaaactagacaaggtaacaactgggttaaaactggtagcataaatcctagtgttataataactggacctagagaaaacattatagatccagaaacttctacgtttaaattaactaacaatacttttgcggcacaagaaggatttggtgctttatcaataatttcaatatcacctagatttatgctaacatatagtaatgcaactaatgatgtaggagagggtagattttctaagtctgaattttgcatggatccaatactaattttaatgcatgaacttaatcatgcaatgcataatttatatggaatagctataccaaatgatcaaacaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatctatagctaaaagacttaatagtataactactgcaaatccttcaagctttaataaatatataggggaatataaacagaaacttattagaaagtatagattcgtagtagaatcttcaggtgaagttacagtaaatcgtaataagtttgttgagttatataatgaacttacacaaatatttacagaatttaactacgctaaaatatataatgtacaaaataggaaaatatatctttcaaatgtatatactccggttacggcgaatatattagacgataatgtttatgatatacaaaatggatttaatatacctaaaagtaatttaaatgtactatttatgggtcaaaatttatctcgaaatccagcattaagaaaagtcaatcctgaaaatatgctttatttatttacaaaattttgtcataaagcaatagatggtagatcattatataataaatgtcaaaatttattcaagaacattaatatcatggccaag

SEQ ID NO: 69 - DNA, 1365 bp, Artificial Sequence
  BoNT/D Light chain-MTDr cDNA Sequence
atgacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcccagacctacttcaaagtatcaaagttattatgatcctagttatttatctactgatgaacaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattatatggaataaatataccatctgataaaaggattcgtccacaagttagcgagggatttttctctcaagatggacccaacgtacaatttgaggaattatatacatttggaggattagatgttgaaataatacctcaaattgaaagatcacaattaagagaaaaagcattaggtcactataaagatatagcgaaaagacttaataatattaataaaactattccttctagttggattagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaggcattatctacctgtatttgcaaatatattagatgataatatttatactataagagatggttttaatttaacaaataaaggttttaatatagaaaattcgggtcagaatatagaaaggaatcctgcactacaaaagcttagttcagaaagtgtagtagatttatttacaaaagtatgtttaagattaacaaaatgtcaaaatttattcaagaacattaatatcatggccaag

SEQ ID NO: 70 - DNA, 1305 bp, Artificial Sequence
  BoNT/E Light chain-MTDr cDNA Sequence
atgccaacaattaatagttttaattataatgatcctgttaataatagaacaattttatatattaaaccaggcggttgtcaacaattttataaatcatttaatattatgaaaaatatttggataattccagagagaaatgtaattggtacaattccccaagattttcttccgcctacttcattgaaaaatggagatagtagttattatgaccctaattatttacaaagtgatcaagaaaaggataaatttttaaaaatagtcacaaaaatatttaatagaataaatgataatctttcaggaaggattttattagaagaactgtcaaaagctaatccatatttaggaaatgataatactccagatggtgacttcattattaatgatgcatcagcagttccaattcaattctcaaatggtagccaaagcatactattacctaatgttattataatgggagcagagcctgatttatttgaaactaacagttccaatatttctctaagaaataattatatgccaagcaatcacggttttggatcaatagctatagtaacattctcacctgaatattcttttagatttaaagataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggtgtcaaaatttattcaagaacattaatatcatggccaag

SEQ ID NO: 71 - DNA, 1347 bp, Artificial Sequence
  BoNT/F Light chain-MTDr cDNA Sequence
atgccagttgcaataaatagttttaattataatgaccctgttaatgatgatacaattttatacatgcagataccatatgaagaaaaaagtaaaaaatattataaagcttttgagattatgcgtaatgtttggataattcctgagagaaatacaataggaacgaatcctagtgattttgatccaccggcttcattaaagaacggaagcagtgcttattatgatcctaattatttaaccactgatgctgaaaaagatagatatttaaaaacaacgataaaattatttaagagaattaatagtaatcctgcagggaaagttttgttacaagaaatatcatatgctaaaccatatttaggaaatgaccacacgccaattgatgaattctctccagttactagaactacaagtgttaatataaaattatcaactaatgttgaaagttcaatgttattgaatcttcttgtattgggagcaggacctgatatatttgaaagttgttgttaccccgttagaaaactaatagatccagatgtagtttatgatccaagtaattatggttttggatcaattaatatcgtgacattttcacctgagtatgaatatacttttaatgatattagtggagggcataatagtagtacagaatcatttattgcagatcctgcaatttcactagctcatgaattgatacatgcactgcatggattatacggggctaggggagttacttatgaagagactatagaagtaaagcaagcacctcttatgatagccgaaaaacccataaggctagaagaatttttaacctttggaggtcaggatttaaatattattactagtgctatgaaggaaaaaatatataacaatcttttagctaactatgaaaaaatagctactagacttagtgaagttaatagtgctcctcctgaatatgatattaatgaatataaagattattttcaatggaagtatgggctagataaaaatgctgatggaagttatactgtaaatgaaaataaatttaatgaaatttataaaaaattatatagttttacagagagtgacttagcaaataaatttaaagtaaaatgtagaaatacttattttattaaatatgaatttttaaaagttccaaatttgttagatgatgatatttatactgtatcagaggggtttaatataggtaatttagcagtaaacaatcgcggacaaagtataaagttaaatcctaaaattattgattccattccagataaaggtctagtagaaaagatcgttaaattttgtaagagcgttattcctagaaaatgtcaaaatttattcaagaacattaatatcatggccaag

SEQ ID NO: 72 - DNA, 1365 bp, Artificial Sequence
  BoNT/G Light chain-MTDr cDNA Sequence
  MISC_FEATURE (20)..(20): n = a, t, c, or g
atgccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaatgtcaaaatttattcaagaacattaatatcatggccaag

SEQ ID NO: 73 - DNA, 1413 bp, Artificial Sequence
  MTD-BoNT/A Light chain cDNA Sequence with hexahistidine
atgaaggccatgatcaatattaacaagttcttaaatcaatgtccctttgtcaacaaacagttcaactacaaggacccagttaatggagtagacatcgcatatatcaagattcccaacgctggccagatgcaacccgttaaggcatttaaaatccataacaaaatctgggttatcccagagcgggataccttcaccaaccccgaggagggcgatctgaaccccccgccggaggcgaagcaggtcccagtgagctactacgatagcacctacctcagcaccgacaacgagaaggacaactacctcaaaggagtcacgaagttgttcgagagaatctactccacagacctcggccgcatgcttctaaccagcattgtgcgtggcattcccttttggggcggctctaccatcgacacagagctgaaggtgatagacaccaactgcatcaacgtaatccagcctgacggcagctaccgaagcgaggagcttaacctggtgatcatcggcccttccgccgatatcatccaattcgagtgcaagagcttcggccacgaggtcctgaacctcacccggaacggctatggaagcacccagtacataagattcagccctgacttcaccttcgggtttgaggagagcttggaggtcgacacaaaccccctgctgggagccgggaagttcgccactgacccagccgtgactctggcacacgagctgatccacgccggtcaccgcctgtacggcatagctataaacccaaacagggtgttcaaagtgaacaccaacgcttactatgaaatgagcggcctggaggtgagcttcgaggagctgagaacgttcgggggacatgatgctaaatttatcgacagcctgcaggagaacgagttcaggctgtactactacaataagttcaaggatatagcgagcactctgaacaaggccaagtccatcgtaggcactactgcatccctccagtatatgaagaatgtgttcaaagagaaatacctgctgagcgaggataccagcggtaagttcagcgtggataagcttaagttcgacaagctgtataagatgctcaccgaaatctacaccgaggataatttcgttaagttcttcaaggtcctgaaccggaagacctacctgaacttcgacaaggccgtgttcaagatcaacatcgtgcctaaagtgaactacaccatctacgacgggtttaacctgaggaacaccaacctggccgctaacttcaacgggcagaacacagagatcaacaacatgaatttcacgaagttgaagaacttcaccggactgtttgagttctacaaattgctgtgtgtgcgcgggatcatcactagcaagaccaagagccttgacaaaggctacaacaagtgactcgagcaccaccaccaccaccactga

SEQ ID NO: 74 - DNA, 1389 bp, Artificial Sequence
  MTD-BoNT/B Light chain cDNA Sequence with hexahistidine
atgaaggccatgatcaatattaacaagttcttaaatcaatgtccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaactcgagcaccaccaccaccaccactga

SEQ ID NO: 75 - DNA, 1413 bp, Artificial Sequence
  MTD-BoNT/C Light chain cDNA Sequence with hexahistidine
atgaaggccatgatcaatattaacaagttcttaaatcaatgtccaataacaattaacaactttaattattcagatcctgttgataataaaaatattttatatttagatactcatttaaatacactagctaatgagcctgaaaaagcctttcgcattacaggaaatatatgggtaatacctgatagattttcaagaaattctaatccaaatttaaataaacctcctcgagttacaagccctaaaagtggttattatgatcctaattatttgagtactgattctgacaaagatacatttttaaaagaaattataaagttatttaaaagaattaattctagagaaataggagaagaattaatatatagactttcgacagatataccctttcctgggaataacaatactccaattaatacttttgattttgatgtagattttaacagtgttgatgttaaaactagacaaggtaacaactgggttaaaactggtagcataaatcctagtgttataataactggacctagagaaaacattatagatccagaaacttctacgtttaaattaactaacaatacttttgcggcacaagaaggatttggtgctttatcaataatttcaatatcacctagatttatgctaacatatagtaatgcaactaatgatgtaggagagggtagattttctaagtctgaattttgcatggatccaatactaattttaatgcatgaacttaatcatgcaatgcataatttatatggaatagctataccaaatgatcaaacaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatctatagctaaaagacttaatagtataactactgcaaatccttcaagctttaataaatatataggggaatataaacagaaacttattagaaagtatagattcgtagtagaatcttcaggtgaagttacagtaaatcgtaataagtttgttgagttatataatgaacttacacaaatatttacagaatttaactacgctaaaatatataatgtacaaaataggaaaatatatctttcaaatgtatatactccggttacggcgaatatattagacgataatgtttatgatatacaaaatggatttaatatacctaaaagtaatttaaatgtactatttatgggtcaaaatttatctcgaaatccagcattaagaaaagtcaatcctgaaaatatgctttatttatttacaaaattttgtcataaagcaatagatggtagatcattatataataaactcgagcaccaccaccaccaccactga

SEQ ID NO: 76 - DNA, 1392 bp, Artificial Sequence
  MTD-BoNT/D Light chain cDNA Sequence with hexahistidine
atgaaggccatgatcaatattaacaagttcttaaatcaatgtacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcccagacctacttcaaagtatcaaagttattatgatcctagttatttatctactgatgaacaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattatatggaataaatataccatctgataaaaggattcgtccacaagttagcgagggatttttctctcaagatggacccaacgtacaatttgaggaattatatacatttggaggattagatgttgaaataatacctcaaattgaaagatcacaattaagagaaaaagcattaggtcactataaagatatagcgaaaagacttaataatattaataaaactattccttctagttggattagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaggcattatctacctgtatttgcaaatatattagatgataatatttatactataagagatggttttaatttaacaaataaaggttttaatatagaaaattcgggtcagaatatagaaaggaatcctgcactacaaaagcttagttcagaaagtgtagtagatttatttacaaaagtatgtttaagattaacaaaactcgagcaccaccaccaccaccactga

SEQ ID NO: 77 - DNA, 1332 bp, Artificial Sequence
  MTD-BoNT/E Light chain cDNA Sequence with hexahistidine
atgaaggccatgatcaatattaacaagttcttaaatcaatgtccaacaattaatagttttaattataatgatcctgttaataatagaacaattttatatattaaaccaggcggttgtcaacaattttataaatcatttaatattatgaaaaatatttggataattccagagagaaatgtaattggtacaattccccaagattttcttccgcctacttcattgaaaaatggagatagtagttattatgaccctaattatttacaaagtgatcaagaaaaggataaatttttaaaaatagtcacaaaaatatttaatagaataaatgataatctttcaggaaggattttattagaagaactgtcaaaagctaatccatatttaggaaatgataatactccagatggtgacttcattattaatgatgcatcagcagttccaattcaattctcaaatggtagccaaagcatactattacctaatgttattataatgggagcagagcctgatttatttgaaactaacagttccaatatttctctaagaaataattatatgccaagcaatcacggttttggatcaatagctatagtaacattctcacctgaatattcttttagatttaaagataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggctcgagcaccaccaccaccaccactga

SEQ ID NO: 78 - DNA, 1374 bp, Artificial Sequence
  MTD-BoNT/F Light chain cDNA Sequence with hexahistidine
atgaaggccatgatcaatattaacaagttcttaaatcaatgtccagttgcaataaatagttttaattataatgaccctgttaatgatgatacaattttatacatgcagataccatatgaagaaaaaagtaaaaaatattataaagcttttgagattatgcgtaatgtttggataattcctgagagaaatacaataggaacgaatcctagtgattttgatccaccggcttcattaaagaacggaagcagtgcttattatgatcctaattatttaaccactgatgctgaaaaagatagatatttaaaaacaacgataaaattatttaagagaattaatagtaatcctgcagggaaagttttgttacaagaaatatcatatgctaaaccatatttaggaaatgaccacacgccaattgatgaattctctccagttactagaactacaagtgttaatataaaattatcaactaatgttgaaagttcaatgttattgaatcttcttgtattgggagcaggacctgatatatttgaaagttgttgttaccccgttagaaaactaatagatccagatgtagtttatgatccaagtaattatggttttggatcaattaatatcgtgacattttcacctgagtatgaatatacttttaatgatattagtggagggcataatagtagtacagaatcatttattgcagatcctgcaatttcactagctcatgaattgatacatgcactgcatggattatacggggctaggggagttacttatgaagagactatagaagtaaagcaagcacctcttatgatagccgaaaaacccataaggctagaagaatttttaacctttggaggtcaggatttaaatattattactagtgctatgaaggaaaaaatatataacaatcttttagctaactatgaaaaaatagctactagacttagtgaagttaatagtgctcctcctgaatatgatattaatgaatataaagattattttcaatggaagtatgggctagataaaaatgctgatggaagttatactgtaaatgaaaataaatttaatgaaatttataaaaaattatatagttttacagagagtgacttagcaaataaatttaaagtaaaatgtagaaatacttattttattaaatatgaatttttaaaagttccaaatttgttagatgatgatatttatactgtatcagaggggtttaatataggtaatttagcagtaaacaatcgcggacaaagtataaagttaaatcctaaaattattgattccattccagataaaggtctagtagaaaagatcgttaaattttgtaagagcgttattcctagaaaactcgagcaccaccaccaccaccactga

SEQ ID NO: 79 - DNA, 1392 bp, Artificial Sequence
  MTD-BoNT/G Light chain cDNA Sequence with hexahistidine
  MISC_FEATURE (59)..(59): n = a, t, c, or g
atgaaggccatgatcaatattaacaagttcttaaatcaatgtccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaactcgagcaccaccaccaccaccactga

SEQ ID NO: 80 - DNA, 1446 bp, Artificial Sequence
  BoNT/A Light chain-MTDr cDNA Sequence with hexahistidine
atgggcagcagccatcatcatcatcatcacagcagcggcctggtgccgcgcggcagccatatgccctttgtcaacaaacagttcaactacaaggacccagttaatggagtagacatcgcatatatcaagattcccaacgctggccagatgcaacccgttaaggcatttaaaatccataacaaaatctgggttatcccagagcgggataccttcaccaaccccgaggagggcgatctgaaccccccgccggaggcgaagcaggtcccagtgagctactacgatagcacctacctcagcaccgacaacgagaaggacaactacctcaaaggagtcacgaagttgttcgagagaatctactccacagacctcggccgcatgcttctaaccagcattgtgcgtggcattcccttttggggcggctctaccatcgacacagagctgaaggtgatagacaccaactgcatcaacgtaatccagcctgacggcagctaccgaagcgaggagcttaacctggtgatcatcggcccttccgccgatatcatccaattcgagtgcaagagcttcggccacgaggtcctgaacctcacccggaacggctatggaagcacccagtacataagattcagccctgacttcaccttcgggtttgaggagagcttggaggtcgacacaaaccccctgctgggagccgggaagttcgccactgacccagccgtgactctggcacacgagctgatccacgccggtcaccgcctgtacggcatagctataaacccaaacagggtgttcaaagtgaacaccaacgcttactatgaaatgagcggcctggaggtgagcttcgaggagctgagaacgttcgggggacatgatgctaaatttatcgacagcctgcaggagaacgagttcaggctgtactactacaataagttcaaggatatagcgagcactctgaacaaggccaagtccatcgtaggcactactgcatccctccagtatatgaagaatgtgttcaaagagaaatacctgctgagcgaggataccagcggtaagttcagcgtggataagcttaagttcgacaagctgtataagatgctcaccgaaatctacaccgaggataatttcgttaagttcttcaaggtcctgaaccggaagacctacctgaacttcgacaaggccgtgttcaagatcaacatcgtgcctaaagtgaactacaccatctacgacgggtttaacctgaggaacaccaacctggccgctaacttcaacgggcagaacacagagatcaacaacatgaatttcacgaagttgaagaacttcaccggactgtttgagttctacaaattgctgtgtgtgcgcgggatcatcactagcaagaccaagagccttgacaaaggctacaacaagtgatgtcaaaatttattcaagaacattaatatcatggccaag

SEQ ID NO: 81 - DNA, 1422 bp, Artificial Sequence
  BoNT/B Light chain-MTDr cDNA Sequence with hexahistidine
atgggcagcagccatcatcatcatcatcacagcagcggcctggtgccgcgcggcagccatatgccagttacaataaataattttaattataatgatcctattgataataataatattattatgatggagcctccatttgcgagaggtacggggagatattataaagcttttaaaatcacagatcgtatttggataataccggaaagatatacttttggatataaacctgaggattttaataaaagttccggtatttttaatagagatgtttgtgaatattatgatccagattacttaaatactaatgataaaaagaatatatttttacaaacaatgatcaagttatttaatagaatcaaatcaaaaccattgggtgaaaagttattagagatgattataaatggtataccttatcttggagatagacgtgttccactcgaagagtttaacacaaacattgctagtgtaactgttaataaattaatcagtaatccaggagaagtggagcgaaaaaaaggtattttcgcaaatttaataatatttggacctgggccagttttaaatgaaaatgagactatagatataggtatacaaaatcattttgcatcaagggaaggcttcgggggtataatgcaaatgaagttttgcccagaatatgtaagcgtatttaataatgttcaagaaaacaaaggcgcaagtatatttaatagacgtggatatttttcagatccagccttgatattaatgcatgaacttatacatgttttacatggattatatggcattaaagtagatgatttaccaattgtaccaaatgaaaaaaaattttttatgcaatctacagatgctatacaggcagaagaactatatacatttggaggacaagatcccagcatcataactccttctacggataaaagtatctatgataaagttttgcaaaattttagagggatagttgatagacttaacaaggttttagtttgcatatcagatcctaacattaatattaatatatataaaaataaatttaaagataaatataaattcgttgaagattctgagggaaaatatagtatagatgtagaaagttttgataaattatataaaagcttaatgtttggttttacagaaactaatatagcagaaaattataaaataaaaactagagcttcttattttagtgattccttaccaccagtaaaaataaaaaatttattagataatgaaatctatactatagaggaagggtttaatatatctgataaagatatggaaaaagaatatagaggtcagaataaagctataaataaacaagcttatgaagaaattagcaaggagcatttggctgtatataagatacaaatgtgtaaaagtgttaaatgtcaaaatttattcaagaacattaatatcatggccaag

SEQ ID NO: 82 - DNA, 1446 bp, Artificial Sequence
  BoNT/C Light chain-MTDr cDNA Sequence with hexahistidine
atgggcagcagccatcatcatcatcatcacagcagcggcctggtgccgcgcggcagccatatgccaataacaattaacaactttaattattcagatcctgttgataataaaaatattttatatttagatactcatttaaatacactagctaatgagcctgaaaaagcctttcgcattacaggaaatatatgggtaatacctgatagattttcaagaaattctaatccaaatttaaataaacctcctcgagttacaagccctaaaagtggttattatgatcctaattatttgagtactgattctgacaaagatacatttttaaaagaaattataaagttatttaaaagaattaattctagagaaataggagaagaattaatatatagactttcgacagatataccctttcctgggaataacaatactccaattaatacttttgattttgatgtagattttaacagtgttgatgttaaaactagacaaggtaacaactgggttaaaactggtagcataaatcctagtgttataataactggacctagagaaaacattatagatccagaaacttctacgtttaaattaactaacaatacttttgcggcacaagaaggatttggtgctttatcaataatttcaatatcacctagatttatgctaacatatagtaatgcaactaatgatgtaggagagggtagattttctaagtctgaattttgcatggatccaatactaattttaatgcatgaacttaatcatgcaatgcataatttatatggaatagctataccaaatgatcaaacaatttcatctgtaactagtaatattttttattctcaatataatgtgaaattagagtatgcagaaatatatgcatttggaggtccaactatagaccttattcctaaaagtgcaaggaaatattttgaggaaaaggcattggattattatagatctatagctaaaagacttaatagtataactactgcaaatccttcaagctttaataaatatataggggaatataaacagaaacttattagaaagtatagattcgtagtagaatcttcaggtgaagttacagtaaatcgtaataagtttgttgagttatataatgaacttacacaaatatttacagaatttaactacgctaaaatatataatgtacaaaataggaaaatatatctttcaaatgtatatactccggttacggcgaatatattagacgataatgtttatgatatacaaaatggatttaatatacctaaaagtaatttaaatgtactatttatgggtcaaaatttatctcgaaatccagcattaagaaaagtcaatcctgaaaatatgctttatttatttacaaaattttgtcataaagcaatagatggtagatcattatataataaatgtcaaaatttattcaagaacattaatatcatggccaag

SEQ ID NO: 83 - DNA, 1425 bp, Artificial Sequence
  BoNT/D Light chain-MTDr cDNA Sequence with hexahistidine
atgggcagcagccatcatcatcatcatcacagcagcggcctggtgccgcgcggcagccatatgacatggccagtaaaagattttaattatagtgatcctgttaatgacaatgatatattatatttaagaataccacaaaataagttaattactacacctgtaaaagcttttatgattactcaaaatatttgggtaataccagaaagattttcatcagatactaatccaagtttaagtaaaccgcccagacctacttcaaagtatcaaagttattatgatcctagttatttatctactgatgaacaaaaagatacatttttaaaagggattataaaattatttaaaagaattaatgaaagagatataggaaaaaaattaataaattatttagtagttggttcaccttttatgggagattcaagtacgcctgaagatacatttgattttacacgtcatactactaatattgcagttgaaaagtttgaaaatggtagttggaaagtaacaaatattataacaccaagtgtattgatatttggaccacttcctaatatattagactatacagcatcccttacattgcaaggacaacaatcaaatccatcatttgaagggtttggaacattatctatactaaaagtagcacctgaatttttgttaacatttagtgatgtaacatctaatcaaagttcagctgtattaggcaaatctatattttgtatggatccagtaatagctttaatgcatgagttaacacattctttgcatcaattatatggaataaatataccatctgataaaaggattcgtccacaagttagcgagggatttttctctcaagatggacccaacgtacaatttgaggaattatatacatttggaggattagatgttgaaataatacctcaaattgaaagatcacaattaagagaaaaagcattaggtcactataaagatatagcgaaaagacttaataatattaataaaactattccttctagttggattagtaatatagataaatataaaaaaatattttctgaaaagtataattttgataaagataatacaggaaattttgttgtaaatattgataaattcaatagcttatattcagacttgactaatgttatgtcagaagttgtttattcttcgcaatataatgttaaaaacaggactcattatttttcaaggcattatctacctgtatttgcaaatatattagatgataatatttatactataagagatggttttaatttaacaaataaaggttttaatatagaaaattcgggtcagaatatagaaaggaatcctgcactacaaaagcttagttcagaaagtgtagtagatttatttacaaaagtatgtttaagattaacaaaatgtcaaaatttattcaagaacattaatatcatggccaag

SEQ ID NO: 84 - DNA, 1365 bp, Artificial Sequence
  BoNT/E Light chain-MTDr cDNA Sequence with hexahistidine
atgggcagcagccatcatcatcatcatcacagcagcggcctggtgccgcgcggcagccatatgccaacaattaatagttttaattataatgatcctgttaataatagaacaattttatatattaaaccaggcggttgtcaacaattttataaatcatttaatattatgaaaaatatttggataattccagagagaaatgtaattggtacaattccccaagattttcttccgcctacttcattgaaaaatggagatagtagttattatgaccctaattatttacaaagtgatcaagaaaaggataaatttttaaaaatagtcacaaaaatatttaatagaataaatgataatctttcaggaaggattttattagaagaactgtcaaaagctaatccatatttaggaaatgataatactccagatggtgacttcattattaatgatgcatcagcagttccaattcaattctcaaatggtagccaaagcatactattacctaatgttattataatgggagcagagcctgatttatttgaaactaacagttccaatatttctctaagaaataattatatgccaagcaatcacggttttggatcaatagctatagtaacattctcacctgaatattcttttagatttaaagataatagtatgaatgaatttattcaagatcctgctcttacattaatgcatgaattaatacattcattacatggactatatggggctaaagggattactacaaagtatactataacacaaaaacaaaatcccctaataacaaatataagaggtacaaatattgaagaattcttaacttttggaggtactgatttaaacattattactagtgctcagtccaatgatatctatactaatcttctagctgattataaaaaaatagcgtctaaacttagcaaagtacaagtatctaatccactacttaatccttataaagatgtttttgaagcaaagtatggattagataaagatgctagcggaatttattcggtaaatataaacaaatttaatgatatttttaaaaaattatacagctttacggaatttgatttagcaactaaatttcaagttaaatgtaggcaaacttatattggacagtataaatacttcaaactttcaaacttgttaaatgattctatttataatatatcagaaggctataatataaataatttaaaggtaaattttagaggacagaatgcaaatttaaatcctagaattattacaccaattacaggtagaggactagtaaaaaaaatcattagattttgtaaaaatattgtttctgtaaaaggcataaggtgtcaaaatttattcaagaacattaatatcatggccaag

SEQ ID NO: 85 - DNA, 1407 bp, Artificial Sequence
  BoNT/F Light chain-MTDr cDNA Sequence with hexahistidine
atgggcagcagccatcatcatcatcatcacagcagcggcctggtgccgcgcggcagccatatgccagttgcaataaatagttttaattataatgaccctgttaatgatgatacaattttatacatgcagataccatatgaagaaaaaagtaaaaaatattataaagcttttgagattatgcgtaatgtttggataattcctgagagaaatacaataggaacgaatcctagtgattttgatccaccggcttcattaaagaacggaagcagtgcttattatgatcctaattatttaaccactgatgctgaaaaagatagatatttaaaaacaacgataaaattatttaagagaattaatagtaatcctgcagggaaagttttgttacaagaaatatcatatgctaaaccatatttaggaaatgaccacacgccaattgatgaattctctccagttactagaactacaagtgttaatataaaattatcaactaatgttgaaagttcaatgttattgaatcttcttgtattgggagcaggacctgatatatttgaaagttgttgttaccccgttagaaaactaatagatccagatgtagtttatgatccaagtaattatggttttggatcaattaatatcgtgacattttcacctgagtatgaatatacttttaatgatattagtggagggcataatagtagtacagaatcatttattgcagatcctgcaatttcactagctcatgaattgatacatgcactgcatggattatacggggctaggggagttacttatgaagagactatagaagtaaagcaagcacctcttatgatagccgaaaaacccataaggctagaagaatttttaacctttggaggtcaggatttaaatattattactagtgctatgaaggaaaaaatatataacaatcttttagctaactatgaaaaaatagctactagacttagtgaagttaatagtgctcctcctgaatatgatattaatgaatataaagattattttcaatggaagtatgggctagataaaaatgctgatggaagttatactgtaaatgaaaataaatttaatgaaatttataaaaaattatatagttttacagagagtgacttagcaaataaatttaaagtaaaatgtagaaatacttattttattaaatatgaatttttaaaagttccaaatttgttagatgatgatatttatactgtatcagaggggtttaatataggtaatttagcagtaaacaatcgcggacaaagtataaagttaaatcctaaaattattgattccattccagataaaggtctagtagaaaagatcgttaaattttgtaagagcgttattcctagaaaatgtcaaaatttattcaagaacattaatatcatggccaag

SEQ ID NO: 86 - DNA, 1425 bp, Artificial Sequence
  BoNT/G Light chain-MTDr cDNA Sequence with hexahistidine
  MISC_FEATURE (80)..(80): n = a, t, c, or g
atgggcagcagccatcatcatcatcatcacagcagcggcctggtgccgcgcggcagccatatgccagttaatataaaaanctttaattataatgaccctattaataatgatgacattattatgatggaaccattcaatgacccagggccaggaacatattataaagcttttaggattatagatcgtatttggatagtaccagaaaggtttacttatggatttcaacctgaccaatttaatgccagtacaggagtttttagtaaagatgtctacgaatattacgatccaacttatttaaaaaccgatgctgaaaaagataaatttttaaaaacaatgattaaattatttaatagaattaattcaaaaccatcaggacagagattactggatatgatagtagatgctataccttatcttggaaatgcatctacaccgcccgacaaatttgcagcaaatgttgcaaatgtatctattaataaaaaaattatccaacctggagctgaagatcaaataaaaggtttaatgacaaatttaataatatttggaccaggaccagttctaagtgataattttactgatagtatgattatgaatggccattccccaatatcagaaggatttggtgcaagaatgatgataagattttgtcctagttgtttaaatgtatttaataatgttcaggaaaataaagatacatctatatttagtagacgcgcgtattttgcagatccagctctaacgttaatgcatgaacttatacatgtgttacatggattatatggaattaagataagtaatttaccaattactccaaatacaaaagaatttttcatgcaacatagcgatcctgtacaagcagaagaactatatacattcggaggacatgatcctagtgttataagtccttctacggatatgaatatttataataaagcgttacaaaattttcaagatatagctaataggcttaatattgtttcaagtgcccaagggagtggaattgatatttccttatataaacaaatatataaaaataaatatgattttgttgaagatcctaatggaaaatatagtgtagataaggataagtttgataaattatataaggccttaatgtttggctttactgaaactaatctagctggtgaatatggaataaaaactaggtattcttattttagtgaatatttgccaccgataaaaactgaaaaattgttagacaatacaatttatactcaaaatgaaggctttaacatagctagtaaaaatctcaaaacggaatttaatggtcagaataaggcggtaaataaagaggcttatgaagaaatcagcctagaacatctcgttatatatagaatagcaatgtgcaagcctgtaatgtacaaatgtcaaaatttattcaagaacattaatatcatggccaag

(19) United States
(12) Patent Application Publication
(10) Pub. No.: US 2024/0408184 A1
(43) Pub. Date: Dec. 12, 2024

(54) COMPOSITION COMPRISING BOTULINUM-DERIVED PEPTIDE FOR PAIN RELIEF

(71) Applicant: BPMED CO., LTD., Seoul (KR)

(72) Inventor: Ki Young YOON, Seoul (KR)

(21) Appl. No.: 18/704,129

(22) PCT Filed: Jun. 2, 2022
(86) PCT No.: PCT/KR2022/007851

§ 371 Date: Apr. 24, 2024

(30) Foreign Application Priority Data
Oct. 25, 2021 (KR) 10-2021-0143024
May 25, 2022 (KR) 10-2022-0064311

(51) Int. Cl. A61K 38/48; A61P 21/02; A61P 25/06

(52) U.S. Cl. CPC A61K 38/4893; A61P 21/02; A61P 25/06; C12Y 304/24069

(57) ABSTRACT

The present invention relates to a pain relief composition including a botulinum-derived peptide. A composition including a botulinum toxin recombinant protein according to the present invention can effectively relieve muscle tension and reduce pain by rapidly delivering a botulinum toxin light chain having a muscle-paralyzing effect to a painful region, and thus it can be effectively used as a substance for preventing, ameliorating, or treating pain.

### TECHNICAL FIELD

The present invention relates to a pain relief composition including a botulinum-derived peptide.

The present invention claims priority based on Korea Patent Application No. 10-2021-0143024 filed on Oct. 25, 2021 and Korea Patent Application No. 10-2022-0064311 filed on May 25, 2022, and all contents disclosed in the specifications and drawings of the above applications are incorporated into the present application.

### BACKGROUND ART

It is generally known that pain plays an important role in protecting the body from danger and recovering damaged tissues. However, pathological pain caused by viral or bacterial infection, severe inflammatory reactions around peripheral nerves, or direct damage to peripheral nerves is harmful to the human body and has a significant impact on the patient's quality of life.

Throughout human history, analgesics have long been developed and used to relieve or numb pain, and these analgesics are generally administered orally. However, oral administration tends to cause problems such as liver damage and kidney damage, and because it must be absorbed through the digestive system before entering the bloodstream, it takes time to exert its efficacy.

To solve these problems of oral administration, parenteral administration methods, such as intravascular injection or intramuscular injection, have been proposed, but these methods are inconvenient, costly, and very difficult to be performed outside hospitals.

Therefore, attempts are being made to administer by topical administration, especially transdermal administration.

Meanwhile, botulinum toxin is a neurotoxin protein produced by Clostridium botulinum, and is reported to inhibit the secretion of acetylcholine and catecholamine, which are neurotransmitters of neurons. There are eight types of botulinum toxin: types A, B, C, D, E, F, G, and H, and types A and B are commercially used.

Botulinum toxin is a neurotoxin protein, and the median lethal dose for humans is 1.3 ng/kg or higher when injected intravenously or intramuscularly, and 10 ng/kg or higher when inhaled, indicating that it is a highly toxic substance. However, when adjusted to an appropriate amount, it can be used as a drug for treating neurological disorders, muscle diseases, hyperhidrosis, and square jaw, etc., and it is most often used for cosmetic purposes such as reducing wrinkles and calf muscles.

Since botulinum toxin is a very large molecule having a molecular weight of 150 kDa with a combined light chain and heavy chain, it is difficult to penetrate the skin and is therefore only used through injections. The toxin lasts for 3 to 6 months and thus the toxin requires regular treatments. Therefore, many studies are being conducted to find another effective delivery means capable of providing user convenience, but the results are still insufficient.

### DISCLOSURE

### Technical Problem

As a result of confirming the pain relief effect of a botulinum toxin recombinant protein (skin-permeating botulinum-derived ingredient peptide) according to the present invention, the present inventors confirmed that it exhibited an excellent pain reduction effect for patients with pain such as a headache and chronic pain, and thereby completed the present invention.

Therefore, an object of the present invention is to provide a pharmaceutical composition for pain relief, comprising a botulinum toxin recombinant protein as an active ingredient, wherein in the botulinum toxin recombinant protein, a cell-penetrating peptide consisting of an amino acid sequence of SEQ ID NO: 1 is fused to one end or both ends of a botulinum toxin light chain.

Another object of the present invention is to provide a quasi-drug composition for pain relief, comprising the botulinum toxin recombinant protein as an active ingredient.

Still another object of the present invention is to provide a composition for external skin application for pain relief, comprising the botulinum toxin recombinant protein as an active ingredient.

Yet another object of the present invention is to provide a cosmetic composition for pain relief, comprising the botulinum toxin recombinant protein as an active ingredient.

However, the technical problems to be solved by the present invention are not limited to the problems mentioned above, and other problems not mentioned may be clearly understood from the description below by those skilled in the art to which the present invention pertains.

### Technical Solution

One aspect of the present invention provides a pharmaceutical composition for pain relief, comprising a botulinum toxin recombinant protein as an active ingredient, wherein in the botulinum toxin recombinant protein, a cell-penetrating peptide consisting of an amino acid sequence of SEQ ID NO: 1 is fused to one end or both ends of a botulinum toxin light chain.

Another aspect of the present invention provides a quasi-drug composition for pain relief, comprising the botulinum toxin recombinant protein as an active ingredient.

Still another aspect of the present invention provides a composition for external skin application for pain relief, comprising the botulinum toxin recombinant protein as an active ingredient.

Yet another aspect of the present invention provides a cosmetic composition for pain relief, comprising the botulinum toxin recombinant protein as an active ingredient.

In one embodiment of the present invention, the botulinum toxin recombinant protein may consist of one or more amino acid sequences selected from the group consisting of SEQ ID NO: 31 to SEQ ID NO: 58, but is not limited thereto.

In another embodiment of the present invention, the botulinum toxin light chain may consist of one or more amino acid sequences selected from the group consisting of SEQ ID NO: 3 to SEQ ID NO: 9, but is not limited thereto.

In still another embodiment of the present invention, the botulinum toxin light chain may further include a hexahistidine tag at one end, but is not limited thereto.

In yet another embodiment of the present invention, the botulinum toxin light chain may be selected from the group consisting of botulinum toxin serotypes A, B, C, D, E, F, and G, but is not limited thereto.

In yet another embodiment of the present invention, the cell-penetrating peptide may be fused to a carboxyl terminus, an amino terminus, or both of the botulinum toxin light chain, but is not limited thereto.

In yet another embodiment of the present invention, the fusion may be achieved by a peptide bond or a covalent bond, but is not limited thereto.

In yet another embodiment of the present invention, the botulinum toxin recombinant protein may relieve muscle tension, but is not limited thereto.

In yet another embodiment of the present invention, the pain may be selected from the somatogenic pain, neuropathic pain, psychogenic pain, heat-induced pain, physical pain, nociception, hyperalgesia, rheumatic pain, headaches, back pain, pelvic pain, myofascial pain, vascular pain, migraines, wound-related pain, inflammatory pain, arthritis pain, diabetic pain, cancer-derived pain, visceral pain, fibromyalgia, postoperative pain, phantom pain, trigeminal neuralgia, postherpetic neuralgia, painful diabetic neuropathy, painful diabetic peripheral neuropathy, diabetic polyneuropathy, sciatic pain, radiculopathy, spinal radiculopathy, myalgia, and lumbar pain, but is not limited thereto.

In yet another embodiment of the present invention, the pharmaceutical composition for pain relief may be for transdermal administration, but is not limited thereto.

In addition, the present invention provides a method of ameliorating pain, comprising administering a composition including the botulinum toxin recombinant protein as an active ingredient to a subject in need thereof.

In addition, the present invention provides a use of a composition comprising the botulinum toxin recombinant protein as an active ingredient for pain relief

In addition, the present invention provides a use of the botulinum toxin recombinant protein for preparing a drug for pain relief.

### Advantageous Effects

A botulinum toxin recombinant protein according to the present invention can transdermally deliver a botulinum toxin light chain having a muscle-paralyzing effect through fusion with a cell-penetrating peptide, effectively relieving muscle tension and reducing pain by conveniently and rapidly delivering the botulinum toxin light chain to a painful region, and thus it can be effectively used as a substance for preventing, ameliorating, or treating pain.

### DESCRIPTION OF DRAWINGS

FIG. 1 is a diagram showing results of comparing the number of headaches by subject/period after using a composition including the botulinum toxin recombinant protein.

FIG. 2 is a diagram showing the results of comparing the rate of reduction in the number of headaches by period after using a composition including the botulinum toxin recombinant protein.

FIG. 3 is a diagram showing the results of comparing the frequency of taking an analgesic by period after using a composition including the botulinum toxin recombinant protein.

FIG. 4 is a diagram showing the results of confirming pain changes for six days after applying a botulinum lotion to patients with chronic pain in order to confirm the pain relief effect of the botulinum toxin recombinant protein (NRS: numerical rating scale; 1 to 4 points: mild pain, 5 to 6 points: moderate pain, and 7 to 10 points: severe pain: hereinafter the same).

FIG. 5 is a diagram showing the results of confirming continued pain reduction for six days after applying a botulinum lotion to patients with fibromyalgia and myalgia in order to confirm the pain relief effect of the botulinum toxin recombinant protein.

FIG. 6 is a diagram showing the results of confirming continued pain reduction for six days after applying a botulinum lotion to patients using a narcotic analgesic in order to confirm the pain relief effect of the botulinum toxin recombinant protein.

FIG. 7 is a diagram schematically showing a skin penetration process of the botulinum toxin recombinant protein.

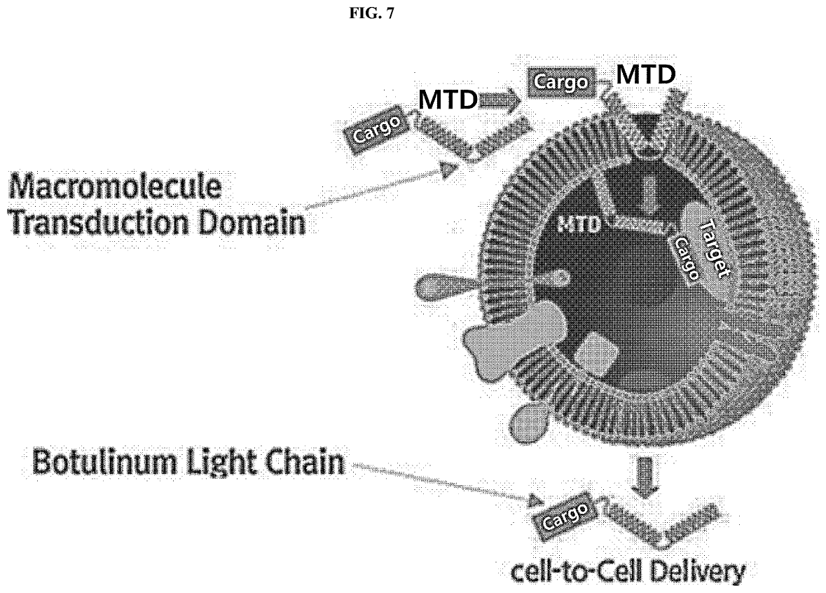

### BEST MODE

As a result of confirming the pain relief effect of a botulinum toxin recombinant protein (skin-penetrating botulinum-derived ingredient peptide) prepared by fusing a cell-penetrating peptide to a botulinum toxin light chain, the present inventors confirmed that it exhibited an excellent pain reduction effect for patients with pain such as a headache and chronic pain, and thereby completed the present invention.

Hereinafter, the present invention will be described in detail.

Botulinum toxin is expressed as a single polypeptide, but after expression, through a reconstitution process, it is divided into a heavy chain (H chain) of about 100 kDa and a light chain (L chain) of about 50 kDa, and the H chain and the L chain are connected by a disulfide bond. The H chain binds to a receptor on a neuron and allows botulinum toxin to enter the inside through endocytosis. After the L chain of botulinum toxin enters a cell, it exits the endosome, enters the cytoplasm, and cleaves a soluble N-ethylmaleimide-sensitive factor attachment protein receptor (SNARE) protein in the cytoplasm, inhibiting acetylcholine secretion and thereby exhibiting a muscle-paralyzing effect. Therefore, inhibition of acetylcholine secretion from neurons is possible with the L chain alone, and the H chain and L chain may function independently.

However, a separated botulinum toxin light chain with a molecular weight of 50 kDa is unable to penetrate the cell membrane, and so it cannot function on its own. In general, in order for a botulinum toxin light chain to be delivered to the cytoplasm of a neuron to exhibit botulinum toxin-specific activity, the help of a botulinum toxin heavy chain of about 100 kDa is essential.

Accordingly, in one embodiment of the present invention, to efficiently deliver a botulinum toxin light chain inside skin and neurons, a composition including a botulinum toxin recombinant protein prepared by fusing a botulinum toxin light chain and a cell-penetrating peptide (Macromolecule Transduction Domain; MTD) as an active ingredient was applied to a painful region of patients with a headache, chronic pain, myalgia, fibromyalgia, or myofascial pain, and as a result, it was confirmed that an excellent pain reduction effect was exhibited.

Therefore, the present invention provides a pharmaceutical composition for pain relief, comprising a botulinum toxin recombinant protein as an active ingredient that allows a botulinum toxin light chain, which is not easily introduced into cells, to be delivered into cells with high efficiency by imparting cell-penetrating properties by fusing a cell-penetrating peptide to the botulinum toxin light chain, and
in the botulinum toxin recombinant protein, a cell-penetrating peptide consisting of an amino acid sequence of SEQ ID NO: 1 is fused to one end or both ends of a botulinum toxin light chain.

In the present invention, “botulinum toxin recombinant protein” includes a cell-penetrating peptide and a botulinum toxin light chain, and refers to a complex formed by a chemical bond such as peptide bond or covalent bond. Specifically, the botulinum toxin recombinant protein according to the present invention is capable of delivering the botulinum toxin light chain into cells with high efficiency by imparting cell-penetrating properties by fusing a cell-penetrating peptide to the botulinum toxin light chain, which is a macromolecule that is not easily introduced into cells, and at this time, the cell-penetrating peptide may be fused to a carboxyl terminus, an amino terminus, or both of the botulinum toxin light chain.

The botulinum toxin recombinant protein according to the present invention may be delivered into cells with high efficiency through the fusion of the botulinum toxin light chain and a cell-penetrating peptide, and the activity and stability of the botulinum toxin light chain are improved to maximize the inherent efficacy of the botulinum toxin in vivo.

The composition of the botulinum toxin recombinant protein (skin-penetrating botulinum-derived ingredient peptide) in the present invention is the same as Korea Patent No. 10-1882461, and all contents disclosed in the above document are incorporated by reference in the present application.

In the present invention, the botulinum toxin recombinant protein may reduce pain by relieving muscle tension and promoting muscle recovery by delivering the botulinum toxin light chain, which has a muscle-paralyzing effect, into cells with high efficiency.

In the present invention, the botulinum toxin recombinant protein may penetrate the skin and access SNAP-25, a component of a SNARE complex, and the botulinum toxin light chain may cleave SNAP-25 to block secretion of acetylcholine, thereby ameliorating pain.

In the present invention, “botulinum toxin” refers to any known type of botulinum toxin, whether subsequently discovered or not, including a variant or a fusion protein produced by bacteria or engineered by a recombinant technique.

In the present invention, the botulinum toxin light chain may be selected from the group consisting of botulinum toxin serotypes A, B, C, D, E, F, and G, and at this time, the botulinum toxin light chain may consist of one or more amino acid sequences selected from the group consisting of SEQ ID NO: 3 to SEQ ID NO: 9. In addition, the botulinum toxin light chain may consist of one amino acid sequence selected from the group consisting of SEQ ID NO: 3 to SEQ ID NO: 9. At this time, the botulinum toxin light chain may be encoded by a polynucleotide consisting of a base sequence selected from the group consisting of SEQ ID NOS: 10 to 16, but is not limited thereto.

In addition, the botulinum toxin light chain may further include a hexahistidine tag at one end. In the present invention, the form further including a hexahistidine tag at one end of the botulinum toxin light chain may consist of an amino acid sequence selected from the group consisting of SEQ ID NO: 17 to SEQ ID NO: 23, and it may be encoded by a base sequence selected from the group consisting of SEQ ID NO: 24 to SEQ ID NO: 30, but is not limited thereto.

In the present invention, the botulinum toxin light chain may alternatively be a botulinum toxin derivative, that is, a compound having botulinum toxin activity but optionally having one or more modifications in a part or sequence. For example, compared to the seven serotypes of the botulinum toxin light chain protein, it may be a form modified in a way that maintains the endopeptidase activity of the light chain while simultaneously enhancing properties or reducing side effects thereof by performing methods such as deletion, modification, replacement, and chimeric fusion on an amino acid sequence. Alternatively, a botulinum toxin light chain or a part of a botulinum toxin light chain produced by recombinant or chemical synthesis may be used.

In the present invention, the botulinum toxin recombinant protein may consist of one or more amino acid sequences selected from the group consisting of SEQ ID NO: 31 to SEQ ID NO: 58, and a polynucleotide encoding the amino acid sequences may be selected from the group consisting of SEQ ID NO: 59 to SEQ ID NO: 86, but is not limited thereto.

In addition, in the present invention, the botulinum toxin recombinant protein may consist of one amino acid sequence selected from the group consisting of SEQ ID NO: 31 to SEQ ID NO: 58.

According to one embodiment of the present invention, the botulinum toxin recombinant protein may preferably consist of an amino acid sequence represented by SEQ ID NO: 45, and a polynucleotide encoding the amino acid sequence may be a nucleotide sequence represented by SEQ ID NO: 73, but is not limited thereto.

In the present invention, the cell-penetrating peptide (MTD) consisting of an amino acid sequence of SEQ ID NO: 1 may be a peptide that may mediate intracellular transport of a biologically active molecule and may have permeability with respect to both human skin keratinocytes and neurons, but is not limited thereto.

The cell-penetrating peptide preferably has no defined enzymatic or therapeutic biological activity, but serves as a carrier allowing for intracellular transport across the cell membrane. It may be attached to an N-terminus or C-terminus and both termini of the cargo to be transferred into the cell, and it may be attached in a forward direction or a reverse direction at each terminus. In addition, the peptide according to the present invention is preferably applied as a monomer, but is not limited thereto, and it may also be used in the form of a dimer or polymer. Furthermore, the peptide according to the present invention may be a peptide including an amino acid sequence of SEQ ID NO: 1 as a minimum unit.

In the present invention, the cell-penetrating peptide may be encoded by a polynucleotide consisting of a base sequence of SEQ ID NO: 2, but is not limited thereto.

In the present specification, “active ingredient” refers to an ingredient that may exhibit desired activity alone or in combination with a carrier that is inactive in itself.

According to one embodiment of the present invention, the most appropriate administration route for the composition according to the present invention to exhibit a pain relief effect is absorption through the skin, and therefore, the composition according to the present invention may preferably be for transdermal administration, but is not limited thereto.

In the present invention, the composition according to the present invention may further include a transdermal absorption enhancer, but is not limited thereto.

The “transdermal absorption enhancer” is a component among emulsifiers that affects skin penetration and is commonly used in transdermal patches. In the present invention, it enhances skin penetration and cellular penetration of the botulinum toxin recombinant protein and may be lecithin, lauryl pyrrolidone, glycerol monooleate, glycerol monolaurate, propylene glycol monolaurate, polyoxyethylene sorbitan monooleate, polyoxyethylene sorbitan monostearate, polyoxyethylene sorbitan monolaurate, sorbitan monooleate, sorbitan monostearate, or sorbitan monolaurate, but is not limited thereto.

In the present invention, “pain” is a general concept that refers to feeling unpleasant emotions due to strong stimulation or physical damage. For the purposes of the present invention, the pain may mean physical pain. Specifically, the pain may be selected from the somatogenic pain, neuropathic pain, psychogenic pain, heat-induced pain, physical pain, nociception, hyperalgesia, rheumatic pain, headaches, back pain, pelvic pain, myofascial pain, vascular pain, migraines, wound-related pain, inflammatory pain, arthritis pain, diabetic pain, cancer-derived pain, visceral pain, fibromyalgia, postoperative pain, phantom pain, trigeminal neuralgia, postherpetic neuralgia, painful diabetic neuropathy, painful diabetic peripheral neuropathy, diabetic polyneuropathy, sciatic pain, radiculopathy, spinal radiculopathy, myalgia, and lumbar pain, but is not limited thereto. According to one embodiment of the present invention, the pain may be headaches, chronic pain, myalgia, fibromyalgia, or myofascial pain.

The term “chronic pain” used in the present invention refers to cases in which the duration and intensity of pain cause a detrimental effect on a patient's function or life or pain persists beyond a normal tissue healing period, which is usually three months.

The term “fibromyalgia” used in the present invention refers to a pain syndrome in tendons, ligaments, fascia, muscles, and soft tissues such as adipose tissue that chronically causes systemic musculoskeletal pain, stiffness, sensory abnormalities, sleep disorders, fatigue, and tender points (parts that hurt when pressed) throughout the body

The term “myofascial pain syndrome” used in the present invention refers to a disease in which muscles become hard and tightly packed together to cause pain and which is caused by a lack of nutrients and oxygen in muscles due to incorrect posture and stress when the muscles around the shoulders and back of the neck are unable to rest and remain tense for a long time.

Proteins (peptides) consisting of amino acid sequences represented by specific sequence numbers described in the present specification are not limited to the corresponding amino acid sequences, and they also include functional equivalents of proteins consisting of the amino acid sequences represented by the specific sequence numbers. “Functional equivalent” refers to a protein having a sequence identity of at least 70%, preferably 80% or more, more preferably 90% or more, and even more preferably 95% or more with an amino acid sequence represented by a specific sequence number as a result of addition, substitution or deletion of amino acids, that is, a protein having a sequence identity of 95% or more and exhibiting substantially the same physiological activity as a protein consisting of an amino acid sequence represented by a specific sequence number. For example, it may include a protein (peptide) having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

A polynucleotide consisting of a base sequence represented by a specific sequence number described in the present specification is not limited to the corresponding base sequence, and variants of the base sequence are included within the scope of the present invention. A nucleic acid molecule of a base sequence of the present invention is a concept including a functional equivalent of the nucleic acid molecule constituting it, for example, variants formed from a nucleic acid molecule in which some base sequences are modified by deletion, substitution, or insertion but are still capable of performing the same function as the nucleic acid. Specifically, a polynucleotide disclosed in the present invention may include a base sequence having a sequence identity of 70% or more, more preferably 80% or more, even more preferably 90% or more, and most preferably 95% or more with a base sequence represented by a specific sequence number. For example, it includes a polynucleotide having a sequence identity of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The “percent sequence identity” of a polynucleotide is confirmed by comparing a comparison region between two optimally aligned sequences, and a part of a polynucleotide sequence in the comparison region may include an addition or deletion (i.e. gap) compared to a reference sequence (including no addition or deletion) for the optimal alignment of two sequences.

In the present invention, “pharmaceutical composition” refers to a composition prepared for the purpose of preventing or treating a disease, and it may be formulated and used in various forms according to conventional methods. For example, it may be formulated into oral dosage forms such as powder, granules, tablets, capsules, suspensions, emulsions, and syrup and formulated and used in the form of external preparations, suppositories, and sterile injection solutions.

The pharmaceutical composition according to the present invention may further include appropriate carriers, excipients, and diluents that are commonly used in preparing pharmaceutical compositions. The excipient may be, for example, one or more selected from the group consisting of diluents, binders, disintegrants, lubricants, adsorbents, humectants, film-coating materials, and controlled-release additives.

The pharmaceutical composition according to the present invention may be formulated and used by conventional methods in the form of as powder, granules, sustained-release granules, enteric-coated granules, solutions, eye drops, elixirs, emulsions, suspensions, spirits, troches, perfumes, limonade, tablets, sustained-release tablets, enteric-coated tablets, sublingual tablets, hard capsules, soft capsules, sustained-release capsules, enteric-coated capsules, pills, tinctures, soft extracts, dry extracts, liquid extracts, injections, capsules, perfusates, plasters, lotions, pastes, sprays, inhalants, patches, sterilized injection solutions, or external preparations such as aerosols, and the external preparations may be formulated as creams, gel, patches, sprays, ointments, plasters, lotions, liniments, pastes, or cataplasmas.

Carriers, excipients, and diluents that may be included in the pharmaceutical composition according to the present invention include lactose, dextrose, sucrose, oligosaccharides, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

In the case of formulation, the composition according to the present invention may be prepared using diluents or excipients such as commonly used fillers, extenders, binders, wetting agents, disintegrants, and surfactants.

As additives for tablets, powder, granules, capsules, pills, and troches according to the present invention, excipients such as corn starch, potato starch, wheat starch, lactose, white sugar, glucose, fructose, di-mannitol, precipitated calcium carbonate, synthetic aluminum silicate, calcium hydrogen phosphate, calcium sulfate, sodium chloride, sodium bicarbonate, purified lanolin, microcrystalline cellulose, dextrin, sodium alginate, methylcellulose, sodium carboxymethylcellulose, kaolin, urea, colloidal silica gel, hydroxypropyl starch, hydroxypropylmethyl cellulose (HPMC) 1928, HPMC 2208, HPMC 2906, HPMC 2910, propylene glycol, casein, calcium lactate, and Primogel; and binders such as gelatin, gum arabic, ethanol, agar powder, cellulose acetate phthalate, carboxymethyl cellulose, calcium carboxymethyl cellulose, glucose, purified water, sodium caseinate, glycerin, stearic acid, sodium carboxymethyl cellulose, sodium methyl cellulose, methyl cellulose, microcrystalline cellulose, dextrin, hydroxycellulose, hydroxypropyl starch, hydroxymethyl cellulose, refined shellac, starch gelatin, hydroxypropyl cellulose, hydroxypropyl methylcellulose, polyvinyl alcohol, and polyvinylpyrrolidone may be used; and disintegrants such as hydroxypropyl methyl cellulose, corn starch, agar powder, methyl cellulose, bentonite, hydroxypropyl starch, sodium carboxymethyl cellulose, sodium alginate, calcium carboxymethyl cellulose, calcium citrate, sodium lauryl sulfate, silicic acid anhydride, 1-hydroxypropyl cellulose, dextran, ion exchange resins, polyvinyl acetate, formaldehyde-treated casein and gelatin, alginic acid, amylose, guar gum, sodium bicarbonate, polyvinylpyrrolidone, calcium phosphate, gelled starch, gum arabic, amylopectin, pectin, sodium polyphosphate, ethyl cellulose, white sugar, magnesium aluminum silicate, D-sorbitol solution, and light anhydrous silicic acid; and lubricants such as calcium stearate, magnesium stearate, stearic acid, hydrogenated vegetable oils, talc, Lycopodium, kaolin, vaseline, sodium stearate, cacao fat, sodium salicylate, magnesium salicylate, polyethylene glycol (PEG) 4000, PEG 6000, liquid paraffin, hydrogen-added soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oils, polyethylene glycol fatty acid ether, sodium chloride, sodium acetate, sodium oleate, DL-leucine, and light anhydrous silicic acid may be used.

As additives for liquid formulations according to the present invention, water, dilute hydrochloric acid, dilute sulfuric acid, sodium citrate, sucrose monostearate, polyoxyethylene sorbitol fatty acid esters (Tween esters), polyoxyethylene monoalkyl ethers, lanolin ethers, lanolin esters, acetic acid, hydrochloric acid, ammonia water, ammonium carbonate, potassium hydroxide, sodium hydroxide, prolamin, polyvinylpyrrolidone, ethyl cellulose, sodium carboxymethyl cellulose or the like may be used.

A solution of white sugar, other sugars, or sweeteners or the like may be used in the syrup according to the present invention, and flavoring agents, colorants, preservatives, stabilizers, suspending agents, emulsifiers, thickening agents or the like may be used as needed.

Purified water may be used in the emulsions according to the present invention, and emulsifiers, preservatives, stabilizers, flavoring agents or the like may be used as needed.

Suspending agents such as acacia, tragacanth, methylcellulose, carboxymethylcellulose, sodium carboxymethylcellulose, microcrystalline cellulose, sodium alginate, HPMC, HPMC 1828, HPMC 2906, HPMC 2910 or the like may be used in the suspensions according to the present invention, and surfactants, preservatives, stabilizers, colorants, and flavoring agents may be used as needed.

The injections according to the present invention may include solvents such as distilled water for injection, 0.9% sodium chloride for injection, Ringer's solution, dextrose for injection, dextrose+sodium chloride for injection, PEG, lactated Ringer's solution, ethanol, propylene glycol, non-volatile oil-sesame oil, cottonseed oil, peanut oil, soybean oil, corn oil, ethyl oleate, isopropyl myristic acid, benzyl benzoate; solubilizing agents such as sodium benzoate, sodium salicylate, sodium acetate, urea, urethane, monoethyl acetamide, butazolidine, propylene glycol, Tweens, nicotinic acid amide, hexamine, and dimethylacetamide: buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), and ethylenediaminetetraacetic acid; antioxidants such as sodium bisulfide 0.1%, sodium formaldehyde sulfoxylate, thiourea, disodium ethylenediaminetetraacetate, and acetone sodium bisulfite; analgesics such as benzyl alcohol, chlorobutanol, procaine hydrochloride, glucose, and calcium gluconate; and suspending agents such as carboxymethyl (CM) sodium, sodium alginate, Tween 80, and aluminum monostearate.

In the suppositories according to the present invention, bases such as cacao oil, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, Lanette wax, glycerol monostearate, Tween or Span, Imhausen, monolene (propylene glycol monostearate), glycerin, Adeps solidus, Buytyrum Tego-G), Cebes Pharma 16, Hexalide Base 95, Cotomar, Hydrocote SP, S-70-XXA, S-70-XX75 (S-70-XX95), Hydrokote 25, Hydrokote 711, Idropostal, Massa estrarium (A, AS, B, C, D, E, I, T), Massa-MF, Massaupol, Masupol-15, Neosupostal-N, Paramount-B, Suposiro (OSI, OSIX, A, B, C, D, H, L), suppositories base type IV (AB, B, A, BC, BBG, E, BGF, C, D, 299), Supostal (N, Es), Wecobi (W, R, S, M, Fs), Tegestor triglyceride base (TG-95, MA, 57) may be used.

Solid preparations for oral administration include tablets, pills, powder, granules, and capsules. These solid preparations are prepared by mixing the extract with at least one excipient, for example, starch, calcium carbonate, sucrose, lactose, gelatin, etc. In addition to simple excipients, lubricants such as magnesium stearate and talc are also used.

Suspensions, oral solutions, emulsions, syrup, and the like correspond to liquid preparations for oral administration, and in addition to the commonly used simple diluents such as water and liquid paraffin, various excipients, for example, wetting agents, sweeteners, flavoring agents, and preservatives may be included. Preparations for parenteral administration include sterilized aqueous solutions, non-aqueous solutions, suspensions, emulsions, freeze-dried preparations, and suppositories. As non-aqueous solvents and suspensions, propylene glycol, polyethylene glycol, vegetable oils such as olive oil, and injectable ester such as ethyl oleate may be used.

The pharmaceutical composition according to the present invention is administered in a pharmaceutically effective amount. In the present invention, “pharmaceutically effective amount” refers to an amount that is sufficient to treat a disease with a reasonable benefit/risk ratio applicable to medical treatment, and an effective dose level may be determined based on factors including the type and severity of a patient's disease, drug activity, sensitivity to drug, administration time, administration route and excretion rate, treatment duration, and drugs used simultaneously, and other factors well known in the medical field.

The pharmaceutical composition according to the present invention may be administered as an individual therapeutic agent or concomitantly with another therapeutic agent, and it may be administered sequentially or simultaneously with conventional therapeutic agents, and it may be administered once or multiple times. It is important to administer an amount that may achieve the maximum effect with the minimum amount without side effects by considering all of the above factors, and this may be easily determined by those skilled in the art to which the present invention pertains.

The pharmaceutical composition of the present invention may be administered to a subject through various routes. All modes of administration are considered, and it may be administered by, for example, oral administration, subcutaneous injection, intraperitoneal administration, intravenous injection, intramuscular injection, paraspinal space (intrathecal) injection, sublingual administration, buccal administration, intrarectal injection, vaginal injection, ocular administration, auricular administration, nasal administration, inhalation, spraying through the mouth or nose, dermal administration, transdermal administration or the like.

The pharmaceutical composition of the present invention is determined according to the type of drug that is an active ingredient along with various relevant factors such as the disease to be treated, administration route, the patient's age, gender, and weight, and the severity of the disease.

In addition, the present invention provides a method of ameliorating pain, comprising administering a composition including the botulinum toxin recombinant protein as an active ingredient to a subject in need thereof.

In addition, the present invention provides a use of a composition comprising the botulinum toxin recombinant protein as an active ingredient for pain relief.

In addition, the present invention provides a use of the botulinum toxin recombinant protein for preparing a drug for pain relief.

In the present invention, “subject” refers to a subject that requires treatment of a disease, and more specifically, human or non-human primates, and mammals such as mice, rats, dogs, cats, horses, and cows.

In the present invention, “administration” refers to providing a predetermined amount of the composition of the present invention to a subject by any appropriate method.

In the present invention, “ameliorating” may refer to any action by which pain symptoms are changed for the better or beneficially changed by administration of the composition according to the present invention, and may also include preventive or therapeutic actions.

In the present invention, “prevention” refers to all actions that suppress or delay the onset of a target disease, and “treatment” refers to all actions that change for the better or beneficially change a target disease and associated metabolic abnormalities thereof by administration of the pharmaceutical composition according to the present invention.

In addition, as another aspect of the present invention, the present invention provides a quasi-drug composition for pain relief, comprising a botulinum toxin recombinant protein as an active ingredient, and in the botulinum toxin recombinant protein, a cell-penetrating peptide consisting of an amino acid sequence of SEQ ID NO: 1 is fused to one end or both ends of a botulinum toxin light chain.

The term “quasi-drug” of the present invention refers to articles with a milder effect than that of pharmaceuticals among articles used for the purpose of diagnosing, treating, ameliorating, alleviating, treating, or preventing diseases in humans or animals. For example, according to the Pharmaceutical Affairs Act, a quasi-drug excludes articles used for pharmaceutical uses and include products used for treatment or prevention of diseases in humans/animals and products that have a mild or no direct effect on the human body.

The quasi-drug composition of the present invention may be prepared as one or more selected from the group consisting of body cleansers, foam, soap, masks, ointments, creams, lotions, essence, and sprays, but is not limited thereto. In addition, the composition may be manufactured in the form of bandages, sanitary napkins or the like, but is not limited thereto.

When the botulinum toxin recombinant protein according to the present invention is used as a quasi-drug additive, the composition may be added as is or used together with another quasi-drug or quasi-drug component, and may be used appropriately according to conventional methods. The mixing amount of an active ingredient may be appropriately determined according to the purpose of use.

In addition, as still another aspect of the present invention, the present invention provides a composition for external skin application for pain relief, comprising a botulinum toxin recombinant protein as an active ingredient, and in the botulinum toxin recombinant protein, a cell-penetrating peptide consisting of an amino acid sequence of SEQ ID NO: 1 is fused to one end or both ends of a botulinum toxin light chain.

The composition for external skin application of the present invention includes a botulinum toxin recombinant protein as an active ingredient and may include a pharmaceutically acceptable carrier. In addition, the composition for external skin application of the present invention may further include adjuvants commonly used in the field of dermatology such as fatty substances, organic solvents, solubilizing agents, thickening and gelling agents, softeners, antioxidants, suspending agents, stabilizers, foaming agents, flavoring agents, surfactants, water, ionic or non-ionic emulsifiers, fillers, sequestering agents and chelating agents, preservatives, vitamins, blocking agents, wetting agents, essential oils, dyes, pigments, hydrophilic or lipophilic active agents, lipid vesicles or any other ingredients commonly used in external skin preparations. In addition, the ingredients may be introduced in amounts commonly used in the field of dermatology.

A dose of the composition for external skin application may vary depending on the weight, age, gender, health conditions of the administration subject, administration period, clearance rate, severity of disease, and the like.

The formulation form of the composition for external skin application includes, but is not limited to, for example, liquid coating agents, sprays, lotions, gels, pastes, ointments, aerosols, powder, patches, creams, roll-ons, and transdermal absorbents.

Pharmaceutically acceptable carriers in the composition for external skin application of the present invention vary depending on the formulation form, but they may include hydrocarbons such as Vaseline, liquid paraffin, and gelled hydrocarbons (Plastibase); animal and vegetable oils such as medium chain fatty acid triglycerides, pork fat, hard fat, and cocoa fat; higher fatty alcohols and fatty acids, and esters thereof such as cetanol, stearyl alcohol, stearic acid, and isopropyl palmitate: water-soluble bases such as polyethylene glycol, 1,3-butylene glycol, glycerol, gelatin, white sugars, and sugar alcohols; emulsifiers such as glycerin fatty acid ester, polyoxyl stearate, and polyoxyethylene hydrogenated castor oil: adhesives such as acrylic acid ester and sodium alginate: propellants such as liquefied petroleum gas and carbon dioxide; and preservatives such as paraoxybenzoic acid esters. Furthermore, in addition to these, stabilizers, fragrances, colorants, pH adjusters, diluents, surfactants, preservatives, antioxidants, and the like may be added as needed. When the composition for external skin application according to the present invention is used, it is preferable to apply it by a conventional method to the skin of an affected region or a region where pain occurs.

In addition, the composition for external skin application according to the present invention may be used by adhering to a solid support such as a wound peeling cover of a conventional bandage. The adhesion may be achieved by saturating the composition of the present invention in a solid support and then dehydrating it. Preferably, the solid support may be first coated with an adhesive to improve the adhesion of the composition of the present invention to the solid support. Examples of the adhesives include polyacrylate and cyanoacrylate.

In addition, as yet another aspect of the present invention, the present invention provides a cosmetic composition for pain relief, comprising a botulinum toxin recombinant protein as an active ingredient, and in the botulinum toxin recombinant protein, a cell-penetrating peptide consisting of an amino acid sequence of SEQ ID NO: 1 is fused to one end or both ends of a botulinum toxin light chain.

The cosmetic composition according to the present invention may be prepared in any formulation form commonly prepared in the art, and may be prepared in the form of adjuvants for topical or systemic application that are conventionally prepared in the field of dermatology together with dermatologically acceptable media, bases, adjuvants, excipients, and the like in addition to the composition of the present invention. For example, it may be prepared in the form of basic cosmetic compositions (toners, creams, essence, cleansers such as cleansing foam and cleansing water, packs, body oils), color cosmetic compositions (foundations, lipsticks, mascaras, makeup bases), hair product compositions (shampoos, conditioners, hair conditioners, hair gels), and soap.

In addition, the cosmetic composition of the present invention may further include adjuvants commonly used in the field of dermatology such as fatty substances, organic solvents, solubilizing agents, thickening and gelling agents, softeners, antioxidants, suspending agents, stabilizers, foaming agents, flavoring agents, surfactants, water, ionic or non-ionic emulsifiers, fillers, sequestering agents and chelating agents, preservatives, vitamins, blocking agents, wetting agents, essential oils, dyes, pigments, hydrophilic or lipophilic active agents, lipid vesicles or any other ingredients commonly used in external skin preparations. In addition, the ingredients may be introduced in amounts commonly used in the field of dermatology.

The excipients may include, but are not limited to, skin emollients, skin penetration enhancers, colorants, flavoring agents, emulsifiers, softeners, thickeners, concentrating agents, gelling agents, suspending agents, stabilizers, foaming agents, surfactants, fillers, and solvents. In addition, the cosmetic composition of the present invention may further include fragrances, pigments, sterilizers, antioxidants, preservatives, adjuvants, and humectants, and may include thickeners, inorganic salts, synthetic polymer materials or the like for the purpose of improving physical properties. For example, when a face wash or soap is prepared using the cosmetic composition of the present invention, it may be easily prepared by adding the extract of the present invention to a conventional face wash or soap base. When a cream is prepared, it may be produced by adding the botulinum toxin recombinant protein of the present invention to a general oil-in-water (O/W) cream base. Here, fragrances, chelating agents, pigments, antioxidants, preservatives or the like and synthetic or natural materials such as proteins, minerals, and vitamins may be added for the purpose of improving physical properties.

Suitable formulation forms of the cosmetic compositions include, for example, solutions, gels, solid or pasty anhydrous products, emulsions obtained by dispersing an oil phase in an aqueous phase, suspensions, microemulsions, microcapsules, microgranules, ionic (liposome) or non-ionic vesicular dispersant forms, creams, toners, lotions, powder, ointments, sprays or conceal sticks. In addition, it may be prepared in the form of foam or in the form of an aerosol composition further including a compressed propellant.

Products to which the cosmetic composition of the present invention may be added include, but are not limited to, skin lotions, skin softeners, skin toners, astringent toners, softening toners, nourishing toners, astringents, lotions, milk lotions, moisturizing lotions, nutritional lotions, body creams, massage creams, nutritional creams, moisturizing creams, hand creams, essence, nutritional essence, packs, soap, shampoos, cleansing foams, cleansing lotions, cleansing creams, body lotions, body cleansers, treatment, beauty essence, emulsions, pressed powder, loose powder, and eye shadows.

The composition according to the present invention may further include extracts such as menthol, shea butter, Scotch pine leaf, aloe vera, peppermint, and rosemary, but is not limited thereto.

### Modes of the Invention

Hereinafter, preferred examples are presented to aid understanding of the present invention. However, the following examples are provided only for easier understanding of the present invention, and the content of the present invention is not limited by the following examples.

### EXAMPLES

### Example 1. Confirming Headache Ameliorating Effect

### 1-1. Experimental Materials and Methods

2.5 cc of a solution containing a botulinum toxin recombinant protein (skin-penetrating botulinum-derived ingredient peptide) prepared according to Korean Patent No. 10-1882461 was directly applied every morning to the painful scalp area and forehead of a total of nine adults men and women (seven women, two men, average age of 36 years old) aged 23 to 53 years old who felt discomfort in their daily lives due to headaches and were taking an analgesic.

Evaluation was carried out by performing a survey with the subjects before the test and then asking the subjects to directly enter the number of headaches and the frequency of taking an analgesic each day when a headache occurred through the ‘Headache Diary App’ provided by the Korean Headache Society. The test period was a total of 12 weeks, and the number of headaches was compared and evaluated based on the information directly entered by the subjects in four intervals: before use, 1 to 4 weeks after use, 5 to 8 weeks after use, and 9 to 12 weeks after use. The frequency of taking an analgesic was compared and evaluated in three intervals: 1 to 4 weeks after use, 5 to 8 weeks after use, and 9 to 12 weeks after use.

### 1-2. Number of Headaches

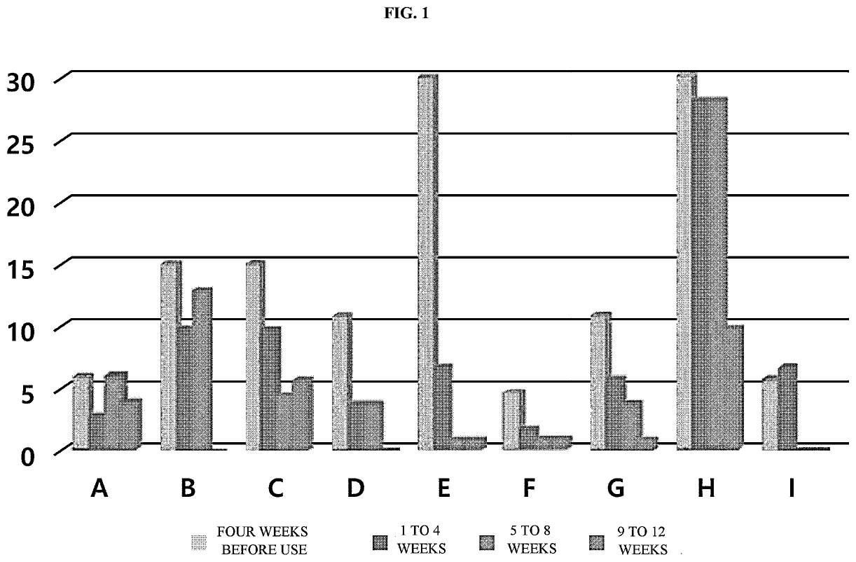

As a result of comparing and evaluating the number of headaches, as shown in FIG. 1, it was confirmed that the headaches was ameliorated in all nine subjects. Specifically, the average number of headaches by period after use was 14.3 in the interval of 4 weeks before use, 8.5 in the interval of 1 to 4 weeks after use, 6.9 in the interval of 5 to 8 weeks after use, and 2.5 in the interval of 9 to 12 weeks after use, which confirmed that the average number of headaches in the subjects decreased by 84% during the interval of 9 to 12 weeks after using the solution containing the skin-penetrating botulinum-derived ingredient peptide compared to the interval of 4 weeks before use.

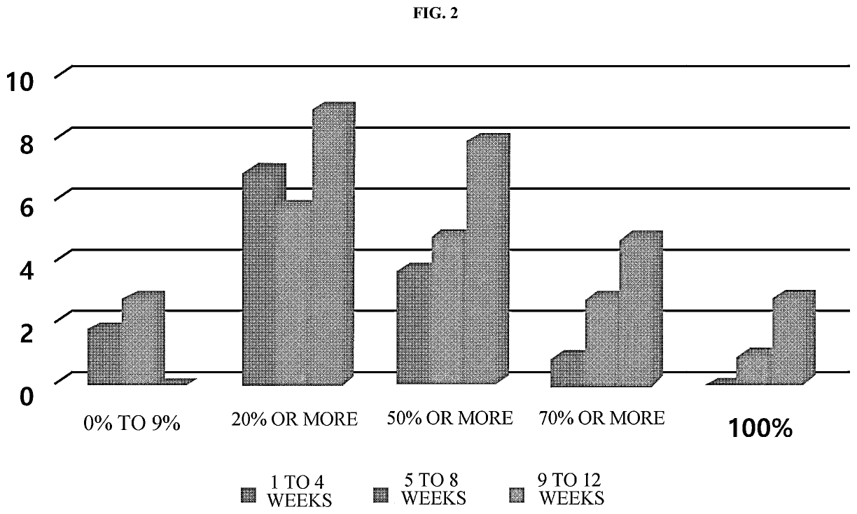

In addition, as shown in FIG. 2, the rate of reduction in the number of headaches by period after use was compared between the first 1 to 4 weeks after use, which correspond to the early stage of use, and the last 9 to 12 weeks after use, and as a result, it was confirmed that two subjects experienced an improvement of more than 20% (7→9); four subjects experienced an improvement of more than 50% (4→8), four subjects experienced an improvement of more than 70% (1→5); and three subjects experienced an improvement of 100% (0→3).

### 1-3. Frequency of Taking Analgesic

As a result of comparing the frequency of taking an analgesic between 1 to 4 weeks and 9 to 12 weeks after use, it was found that the frequency of taking an analgesic decreased in eight of nine subjects and increased by one in one subject.

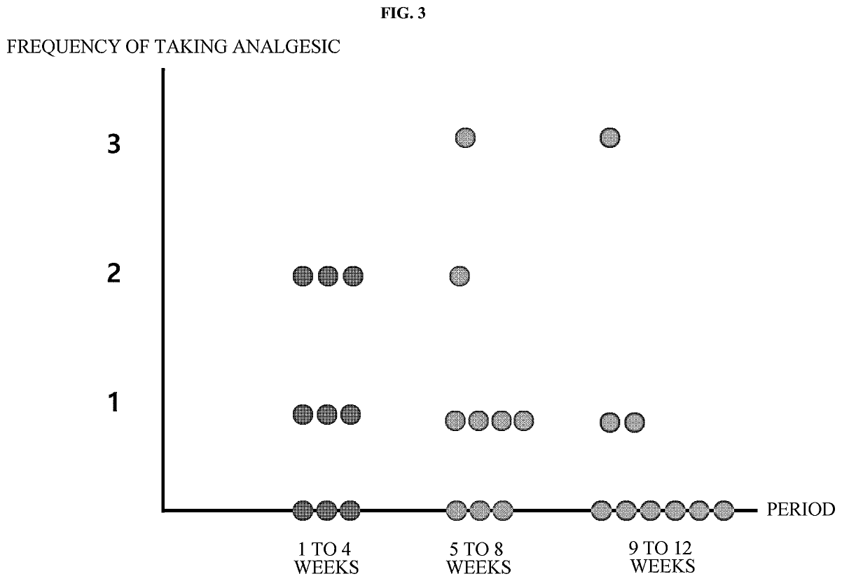

As a result of comparing the frequency of taking an analgesic by period after use, as shown in FIG. 3, in the interval of 1 to 4 weeks after use, three subjects took an analgesic twice, three subjects took it once, and three subjects took it zero times, and in the interval of 5 to 8 weeks after use, one subject took an analgesic three times, two subjects took it twice, four subjects took it once, and three subjects took it zero times, indicating that the frequency of taking an analgesic increased by one in one subject and decreased by one in one subject. It was confirmed that in the interval of 9 to 12 weeks after use, one subject took an analgesic three times, no subject took it twice, two subjects took it once, and six subjects took it zero times, indicating that the number of subjects who did not need to take an analgesic increased by three compared to the interval of 1 to 4 weeks after use, which corresponds to the early stage of use.

From the above results, when the skin-penetrating botulinum-derived ingredient peptide, which exhibits a wrinkle-ameliorating effect similar to a botulinum toxin injection, was applied to the head and neck, the overall number of headaches and the overall frequency of taking an analgesic decreased, and thus it was judged that there would be a headache-ameliorating effect.

### Example 2. Confirmation of Pain Relief Effect

### 2-1. Experimental Materials and Methods

This experiment was performed with 24 adults aged 30 to 75 years who were patients with chronic pain, fibromyalgia, myofascial pain, or myalgia by applying the skin-penetrating botulinum-derived ingredient peptide to their skin to confirm whether the peptide may reduce pain by relieving muscle tension and reduce muscle spasm pain and the degree of ameliorating chronic pain caused by repetitive movement and mechanical shock of the skin-penetrating botulinum-derived ingredient peptide.

The test subjects were selected to meet one of the three criteria in Table 1 below, and they were allowed to directly apply an appropriate amount of a botulinum lotion containing the skin-penetrating botulinum-derived ingredient peptide to the painful region twice daily.

TABLE 1

| | No narcotic | | |
|---|---|---|---|
| | analgesics used | | |
| | (use of antipyretic | Weak narcotic | Strong narcotic |
| Criteria | analgesics included) | analgesics used | analgesics used |
| Durg | Tylenol, NSAID | tramadol, codeine | morphine, |
| examples | | | oxycodone, |
| | | | hydrocodone, |
| | | | fentanyl, tapentadol |

For analysis, the 24 subjects were divided into three groups as follows:
Group A: Chronic pain patients
Group B: Fibromyalgia and myalgia patients
Group C: Narcotic analgesic use group (Group C has sample overlap with Groups A and B).

### 2-2. Group A

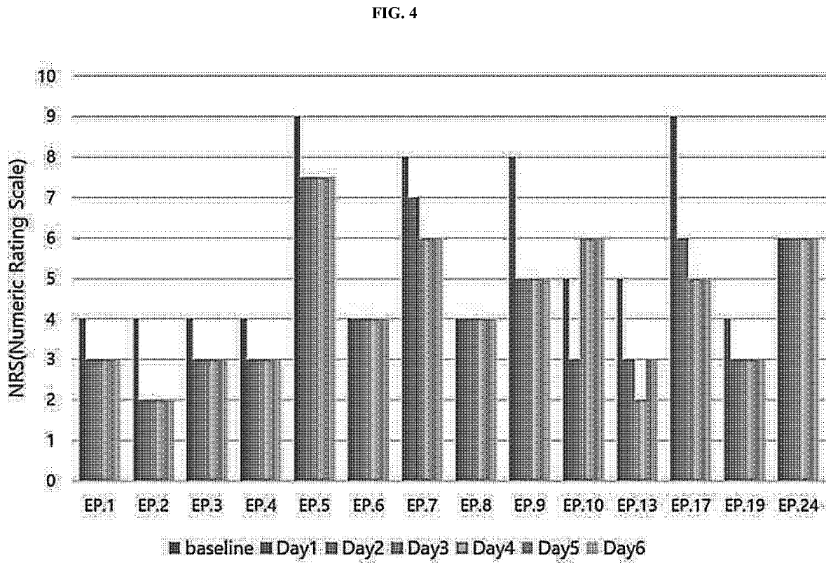

As a result of confirming pain changes over 6 days after using the botulinum lotion in 14 chronic pain patients (Patients satisfying each of the criteria in Table 1 were appropriately mixed), as shown in FIG. 4, it was confirmed that pain was reduced in 78.57% of the patients after using the botulinum lotion (pain reduced in 11 of 14 subjects). Pain remained reduced while using the botulinum lotion. An immediate pain reduction effect was exhibited immediately after the botulinum lotion was used, and it was confirmed that the pain reduction effect was generally maintained while applying the lotion.

### 2-3. Group B

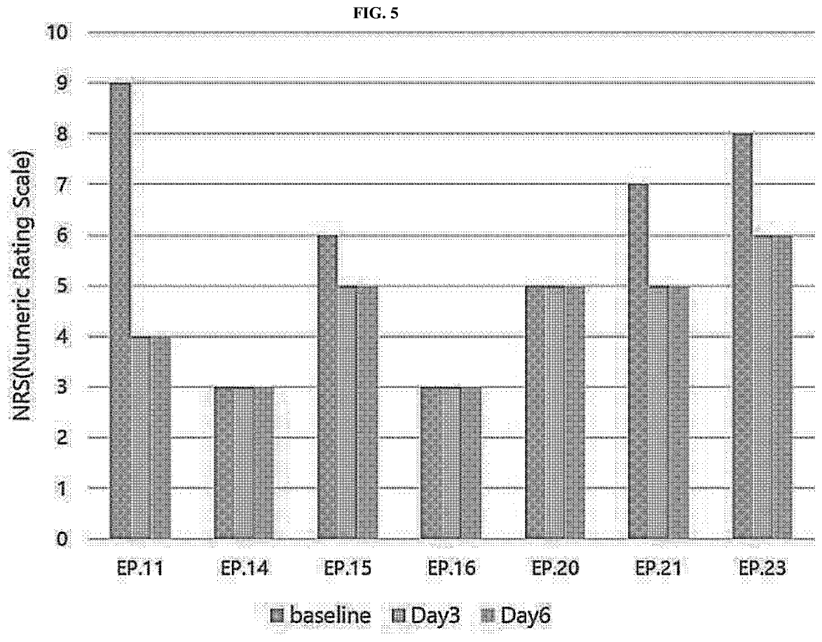

As a result of confirming whether pain reduction continued after the botulinum lotion was used in seven patients with fibromyalgia and myalgia (Patients satisfying each of the criteria in Table 1 were appropriately mixed), it was confirmed that pain was reduced in 71.43% of the patients immediately after using the botulinum lotion (pain reduced in five of seven subjects, now shown in the figure), and as shown in FIG. 5, the pain reduction state was maintained in 57.14% of the patients while applying the lotion (pain reduction state maintained in four of seven subjects). Generally, it was found that an immediate pain reduction effect was experienced immediately after using the botulinum lotion, and the effect was maintained while applying the botulinum lotion.

### 2-4. Group C

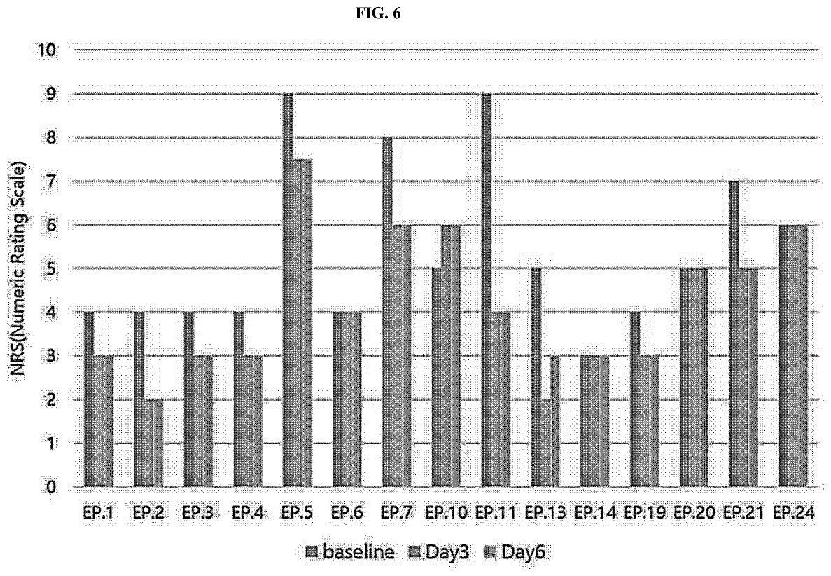

As a result of confirming whether pain reduction continued after the botulinum lotion was used in 15 patients using a narcotic analgesic, as shown in FIG. 6, 80% of the patients responded that pain was immediately relieved 6 (not shown in the figure), and as shown in FIG. 6, 66.67% of the patients (10 of 15 subjects) responded that there was a continued pain relief effect while using the botulinum lotion. It was found that immediately after using the botulinum lotion, an immediate pain reduction effect was generally experienced, and the effect was maintained while applying the botulinum lotion.

The description of the present invention described above is for illustrative purposes, and those skilled in the art will understand that the present invention can be easily modified into other specific forms without changing the technical idea or essential features of the present invention. Therefore, the examples described above should be understood in all respects as illustrative and not restrictive.

### INDUSTRIAL APPLICABILITY

The botulinum toxin recombinant protein according to the present invention can transdermally deliver a botulinum toxin light chain having a muscle-paralyzing effect through fusion with a cell-penetrating peptide, effectively relieving muscle tension and reducing pain by conveniently and rapidly delivering the botulinum toxin light chain to a painful region, and thus it can be effectively used as a substance for preventing, ameliorating, or treating pain. Therefore, the botulinum toxin recombinant protein according to the present invention has industrial applicability.

1. A method of ameliorating pain, comprising administering a therapeutically effective amount of a composition comprising a botulinum toxin recombinant protein as an active ingredient to a subject in need thereof,
wherein in the botulinum toxin recombinant protein, a cell-penetrating peptide consisting of an amino acid sequence of SEQ ID NO: 1 is fused to one end or both ends of a botulinum toxin light chain.

2. The method according to claim 1, wherein the botulinum toxin recombinant protein consists of one or more amino acid sequences selected from the group consisting of SEQ ID NO: 31 to SEQ ID NO: 58.

3. The method according to claim 1, wherein the botulinum toxin light chain consists of one or more amino acid sequences selected from the group consisting of SEQ ID NO: 3 to SEQ ID NO: 9.

4. The method according to claim 1, wherein the botulinum toxin light chain further includes a hexahistidine tag at one end.

5. The method according to claim 1, wherein the botulinum toxin light chain is selected from the group consisting of botulinum toxin serotypes A, B, C, D, E, F, and G.

6. The method according to claim 1, wherein the cell-penetrating peptide is fused to a carboxyl terminus, an amino terminus, or both of the botulinum toxin light chain.

7. The method according to claim 1, wherein the fusion is achieved by a peptide bond or a covalent bond.

8. The method according to claim 1, wherein the botulinum toxin recombinant protein relieves muscle tension.

9. The method according to claim 1, wherein the pain is selected from the group consisting of mechanical pain, thermal pain, chronic pain, acute pain, hyperalgesic pain, somatogenic pain, neuropathic pain, psychogenic pain, heat-induced pain, physical pain, nociception, hyperalgesia, rheumatic pain, headaches, back pain, pelvic pain, myofascial pain, vascular pain, migraines, wound-related pain, inflammatory pain, arthritis pain, diabetic pain, cancer-derived pain, visceral pain, fibromyalgia, postoperative pain, phantom pain, trigeminal neuralgia, postherpetic neuralgia, painful diabetic neuropathy, painful diabetic peripheral neuropathy, diabetic polyneuropathy, sciatic pain, radiculopathy, spinal radiculopathy, myalgia, and lumbar pain.

10. The method according to claim 1, wherein the pharmaceutical composition is for transdermal administration.

11-16. (canceled)

17. The method according to claim 1, wherein the composition is a pharmaceutical composition, a quasi-drug composition, a composition for external skin application, or a cosmetic composition.
